(19) United States
(12) Patent Application Publication
(10) Pub. No.: US 2026/0124077 A1
(43) Pub. Date: May 7, 2026

(54) TUBE DEPLOYING SYSTEM WITH VISUAL INDICATOR

(71) Applicant: Preceptis Medical, Inc., Golden Valley, MN (US)

(72) Inventors: Rudolf Andreas Deibel, Eden Prairie, MN (US); Keith J. Leland, Medina, MN (US)

(21) Appl. No.: 19/375,369

(22) Filed: Oct. 31, 2025

Related U.S. Application Data
(60) Provisional application No. 63/715,086, filed on Nov. 1, 2024.

(51) Int. Cl. A61F 11/20

(52) U.S. Cl. CPC A61F 11/202

(57) ABSTRACT

A tube deploying system includes a handle having a pull mechanism and a nose assembly. The nose assembly includes a hollow cutting sheath, a positioning rod located within the hollow cutting sheath, an actuation member coupling the pull mechanism to the cutting sheath and a visual indicator coupled to the positioning rod. In an undeployed configuration, the cutting sheath is configured to pierce and manually advance a distal cutting edge through a membrane of a patient until an end of the visual indicator that protrudes through a slot in the cutting sheath is adjacent to the membrane. In a deployed configuration, the actuation member is configured to retract the cutting sheath from around the ventilation tube and along the visual indicator and the positioning rod to leave the ventilation tube in a deployed state in the membrane of the patient.

### CROSS-REFERENCE TO RELATED APPLICATION

The present application is based on and claims the benefit of U.S. provisional patent application Serial No. 63/715,086, filed November 1, 2024, the content of which is hereby incorporated by reference in its entirety.

### SUMMARY

A tube deploying system includes a handle having a pull mechanism. The nose assembly includes a hollow cutting sheath having a distal cutting edge and a cutting sheath slot that extends from the distal cutting edge to a terminating end. In an undeployed configuration, the hollow cutting sheath is configured to house a ventilation tube that has a medial end and a lateral end in an undeployed state. The nose assembly further includes a positioning rod having a distal end located within the hollow cutting sheath, an actuation member coupling the pull mechanism to the hollow cutting sheath and a visual indicator coupled to the positioning rod and includes a base and a tab, wherein the tab protrudes through the cutting sheath slot and outwardly from an outer surface of the hollow cutting sheath and distally to an end that is a spaced distance from the distal cutting edge of the hollow cutting sheath in an undeployed configuration. In the undeployed configuration, the hollow cutting sheath is configured to pierce a membrane of a patient with the distal cutting edge and be manually advanced through the membrane until the end of the visual indicator is adjacent to the membrane. In a deployed configuration the actuation member is configured to retract the hollow cutting sheath from around the ventilation tube and along the visual indicator and the positioning rod to leave the ventilation tube in a deployed state in the membrane of the patient.

A tube deploying system includes a ventilation tube having a medial end and a lateral end and a hollow cutting sheath having a distal cutting edge and a cutting sheath slot that extends from the distal cutting edge to a terminating end. The hollow sheath is configured to house the ventilation tube. The tube deploying system further includes a positioning rod having a distal end located within the hollow cutting sheath and being adjacent the ventilation tube, an actuation member coupled to the hollow cutting sheath and a visual indicator including a base and a tab that protrudes from the base. The base is located and fixed internal to the positioning rod. The tab protrudes through the cutting sheath slot and protrudes outwardly from an outer surface of the hollow cutting sheath. The tab includes a first end and a second end that is located distally from the base. The hollow cutting sheath is configured to pierce a membrane of a patient with the distal cutting edge and be manually advanced into the membrane up to the second end of the visual indicator. The actuation member is configured to retract the hollow cutting sheath from around the ventilation tube and along the visual indicator and the positioning rod to deploy and leave the ventilation tube in the membrane.

A method of deploying a ventilation tube includes providing a handle having a pull mechanism and a nose assembly coupled to the handle. The nose assembly includes a hollow cutting sheath having a distal cutting edge and a cutting sheath slot that extends from the distal cutting edge to a terminating end, a positioning rod having a distal end located within the hollow cutting sheath, an actuation member coupling a pull mechanism to the hollow cutting sheath, a ventilation tube having a medial end and a lateral end and being housed in the hollow cutting sheath and a visual indicator that is coupled to the positioning rod and protrudes through the cutting sheath slot and outwardly from an outer surface of the hollow cutting sheath and distally to an end of the visual indicator that is a spaced distance from the distal cutting edge of the hollow cutting sheath.. The nose assembly is manually advanced in an orifice of a patient, pierces a membrane of the patient with the distal cutting edge of the hollow cutting sheath and inserts the hollow cutting sheath through the membrane for the spaced distance until the end of the visual indicator is adjacent the membrane. The pull mechanism is manually moved to retract the hollow cutting sheath from around the ventilation tube to deploy the ventilation tube and thereby leave the deployed ventilation tube in the membrane.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

FIG. 1 is a simplified diagram of an ear.

FIG. 2 is an exemplary perspective view of a tube deploying system according to an embodiment.

FIG. 3 is a perspective view of an end portion of a nose assembly of a tube deploying system in an undeployed configuration according to an embodiment.

FIG. 4 is a medial end view of FIG. 3.

FIG. 5 is an exploded perspective view of FIG. 3.

FIG. 6 illustrates a side view of FIG. 3 after incising a tympanic membrane.

FIG. 7 is a perspective view of FIG. 3 in a deployed configuration according to an embodiment.

FIG. 8 illustrates a medial end view of FIG. 7.

FIG. 9 illustrates a side view of FIG. 7 with the tube deployed in the tympanic membrane.

FIG. 10-16 are perspective and orthogonal views of a visual indicator according to another embodiment.

FIGS. 17 is a top view of a positioning rod according to another embodiment.

FIG. 18 is a bottom view of FIG. 17.

FIG. 19 is an enlarged perspective view of a distal end of the positioning rod in FIGS. 17-18.

FIG. 20 is a perspective view of an end portion of a nose assembly of a tube deploying system using the visual indicator in FIGS. 10-16 and the positioning rod in FIGS. 17-19 in an undeployed configuration according to another embodiment.

FIG. 21 is a medial end view of FIG. 20.

FIG. 22 is an exploded view of FIG. 20.

FIG. 23 is an enlarged perspective view of the distal end of the positioning rod of FIGS. 17-19 with the visual indicator of FIGS. 10-16 assembled thereto and all other components deleted from view.

FIG. 24 is a side view of FIG. 20 after incising a tympanic membrane.

FIG. 25 is a perspective view of FIG. 20 in a deployed configuration according to an embodiment.

FIG. 26 is a medial end view of FIG. 25.

FIG. 27 is a side view of FIG. 25 with the tube deployed in the tympanic membrane.

FIG. 28 is a perspective view of a ventilation tube according to another embodiment.

FIG. 29 is a perspective view of an end portion of a nose assembly with the tube of FIG. 28 in a deployed configuration according to an embodiment.

FIG. 30 is a medial end view of FIG. 29.

FIG. 31 is a side view of FIG. 29 with the tube deployed in the tympanic membrane.

FIG. 32 is a perspective view of a ventilation tube according to another embodiment.

FIG. 33 is a perspective view of an end portion of a nose assembly with the of FIG. 32 in a deployed configuration according to an embodiment.

FIG. 34 is a medial end view of FIG. 33.

FIG. 35 is a side view of FIG. 33 with the tube deployed in the tympanic membrane.

FIG. 36 is a perspective view of an end portion of a nose assembly of a tube deploying system in an undeployed configuration according to an embodiment.

FIG. 37 is a medial end view of FIG. 36.

FIG. 38 is an exploded perspective view of FIG. 36.

FIG. 39 is a side view of FIG. 36 after incising a tympanic membrane.

FIG. 40 is a perspective view of FIG. 36 in a deployed configuration according to an embodiment.

FIG. 41 is a medial end view of FIG. 40.

FIG. 42 is a side view of FIG. 40 with the tube deployed in the tympanic membrane.

FIG. 43 is a perspective view of an end portion of a nose assembly of a tube deploying system in an undeployed configuration according to an embodiment.

FIG. 44 is a medial end view of FIG. 43.

FIG. 45 is an exploded perspective view of FIG. 43.

FIG. 46 is a side view of FIG. 43 after incising a tympanic membrane.

FIG. 47 is a perspective view of FIG. 43 in a deployed configuration according to an embodiment.

FIG. 48 is a medial end view of FIG. 47.

FIG. 49 is a side view of FIG. 47 with the tube deployed in the tympanic membrane.

FIG. 50 is a perspective view of an end portion of a nose assembly of a tube deploying system in an undeployed configuration according to an embodiment.

FIG. 51 is a medial end view of FIG. 50.

FIG. 52 is an exploded perspective view of FIG. 50.

FIG. 53 is a side view of FIG. 50 after incising a tympanic membrane.

FIG. 54 is a perspective view of FIG. 50 in a deployed configuration according to an embodiment.

FIG. 55 is a medial end view of FIG. 54.

FIG. 56 is a side view of FIG. 54 with the tube deployed in the tympanic membrane.

FIG. 57 is a perspective view of an end portion of a nose assembly of a tube deploying system in an undeployed configuration according to an embodiment.

FIG. 58 is a medial end view of FIG. 57.

FIG. 59 is an exploded perspective view of FIG. 57.

FIG. 60 is a side view of FIG. 57 after incising a tympanic membrane.

FIG. 61 is a perspective view of FIG. 57 in a deployed configuration according to an embodiment.

FIG. 62 is a medial end view of FIG. 61.

FIG. 63 is a side view of FIG. 61 with the tube deployed in the tympanic membrane.

FIG. 64 is a perspective view of an end portion of a nose assembly of a tube deploying system in an undeployed configuration according to an embodiment.

FIG. 65 is a medial end view of FIG. 64.

FIG. 66 is an exploded perspective view of FIG. 64.

FIG. 67 is a side view of FIG. 64 after incising a tympanic membrane.

FIG. 68 is a perspective view of FIG. 64 in a deployed configuration according to an embodiment.

FIG. 69 is a medial end view of FIG. 68.

FIG. 70 is a side view of FIG. 68 with the tube deployed in the tympanic membrane.

FIG. 71 is a perspective view of an end portion of a nose assembly of a tube deploying system in an undeployed configuration according to an embodiment.

FIG. 72 is a medial end view of FIG. 71.

FIG. 73 is an exploded perspective view of FIG. 71.

FIG. 74 is a side view of FIG. 71 after incising a tympanic membrane.

FIG. 75 is a perspective view of FIG. 71 in a deployed configuration according to an embodiment.

FIG. 76 is a medial end view of FIG. 75.

FIG. 77 is a side view of FIG. 75 with the tube deployed in the tympanic membrane.

### DETAILED DESCRIPTION

Ventilation tubes, such as pressure equalization (PE) ear tubes or tympanostomy tubes, are hollow devices inserted into tissue or membranes of a human body to enable drainage and provide air flow. For example, an ear tube inserted into a tympanic membrane (eardrum) provides drainage of the middle ear, allows air to flow into the middle ear and prevents the buildup of fluids behind the eardrum. In one embodiment, a ventilation tube includes a material that allows the tube to remain in a deformed state before deployment into a body. After insertion through a target membrane, the tube is allowed to re-form, unfold, deploy and anchor in place. The tube deploying system that includes an insertion device or inserter that places the ventilation tube in the membrane allows for minimally invasive ventilation tube placement, which reduces the pain, cost and risks associated with conventional procedures and devices.

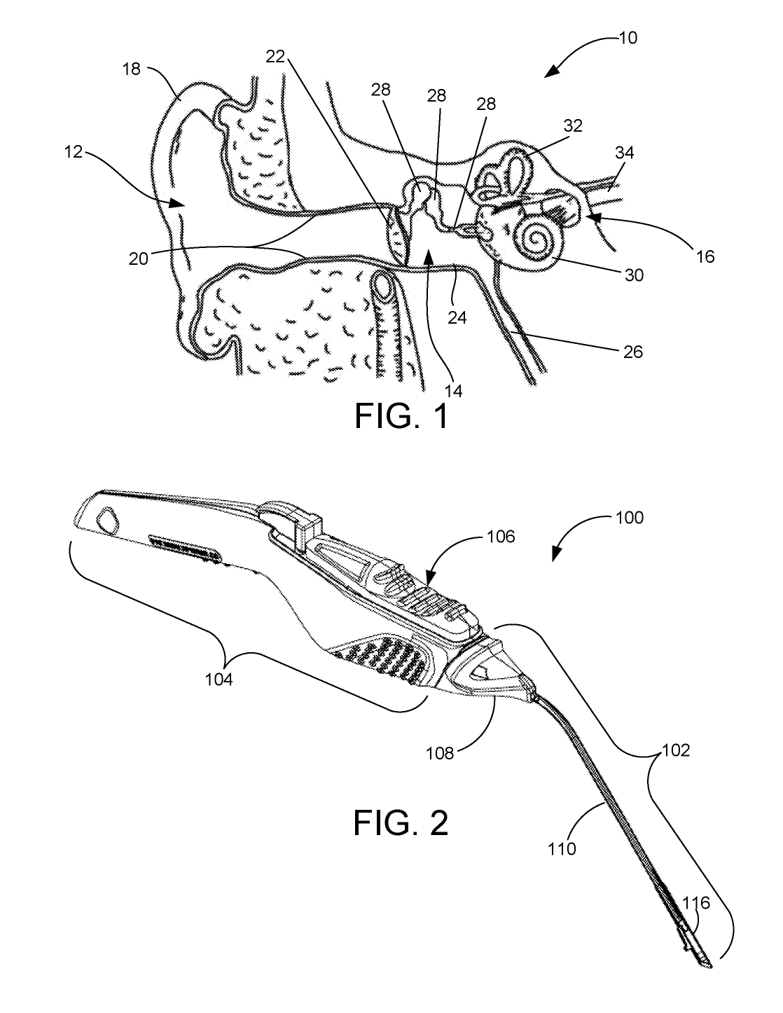

FIG. 1 illustrates a system of organs in an ear 10 of a body that enables a person to detect sound. Ear 10 is able to change sound pressure waves into a signal of nerve impulses to be processed by the brain. Ear 10 includes an outer ear 12, a middle ear 14 and an inner ear 16. Outer ear 12 collects sound and includes the pinna 18, the ear canal 20 and an outer most layer of the ear drum or tympanic membrane (TM) 22. Pinna 18 helps direct sound through ear canal 20 to TM 22. Middle ear 14 includes an air-filled cavity 24 having an opening for the Eustachian tube 26 that is located behind TM 22. Middle ear 14 also includes ossicles 28. Inner ear 16 includes the fluid-filled cochlea 30 and the semicircular canals 32. Cochlea 30 is the auditory portion of the inner ear, while semicircular canals 32 are attuned to both gravity and motion. The ossicles bones 28 transmit sound from the tympanic membrane 22 to cochlea 30. Fluid in cochlea 30 moves in response to the vibrations coming from middle ear 14. The motion of the fluid is converted to electrical impulses, which travel along the auditory nerve 34 to structures in the brainstem for further processing. Eustachian tube 26 couples cavity 24 of middle ear 14 to the nose and mouth of a human. In a normal state, Eustachian tube 26 is collapsed. However, Eustachian tube 26 can open and close to equalize pressure in cavity 24.

An infection of the middle ear 14 can result in a buildup of fluid and increased pressure in cavity 24 causing severe pain. Children are often prone to infections of middle ear 14 because of their underdeveloped Eustachian tube 26. A myringotomy is a surgical procedure in which an incision is created in TM 22 to relieve pressure caused by the excessive buildup of fluid due to an infection of the middle ear 14. If a patient requires a myringotomy, this generally suggests that Eustachian tube 26 is either partially or completely obstructed and is not able to perform its proper functions.

In some cases, besides making an incision in TM 22, a ventilation tube is placed into the incision or opening. Insertion of a ventilation tube or pressure equalizing (PE) ear tube into TM 22 can allow external ventilation of or drainage of middle ear 14 for an extended period of time. However, in the confined space of ear canal 20, especially an ear canal of a child, insertion of a ventilation tube may be difficult. In one example, the incision made in TM 22 is often made larger than the cross-section area of the ventilation tube. In such an example, the device will fall out much earlier than desired. In another example, the tube may be over inserted or placed too far inside middle ear 14 or not far enough. During manual insertion, it is important for clinicians to have visual and/or haptic feedback so as to confirm that the tube is properly inserted into the membrane. Inserting the tube manually into the incision in the tympanic membrane 22 requires manipulation of the freshly made incision, which can cause discomfort or pain in the patient. An insertion device that alleviates these disadvantages can greatly enhance patient comfort as well as reduce procedural time and undue injury to TM 22, while simultaneously simplifying the procedure for physicians.

FIG. 2 illustrates a perspective view of a tube deploying system 100 according to an embodiment. Tube deploying system 100 combines the steps of making an incision in a membrane, such as a myringotomy in a tympanic membrane or eardrum, positioning a tube (e.g., ventilation tube such as a pressure equalization (PE) tube or tympanostomy tube) and placing or inserting the tube in a patient’s membrane. Tube deploying system 100 includes a nose assembly 102 connected to a handle 104 and a pull mechanism 106 that both protrudes from the handle and is housed inside the handle. Nose assembly 102 includes a nose 108, a positioning rod 110 extending from the nose, a hollow cutting sheath 116 and an actuation member (hidden from view in FIG. 2) that couples the cutting sheath 116 to pull mechanism 106.

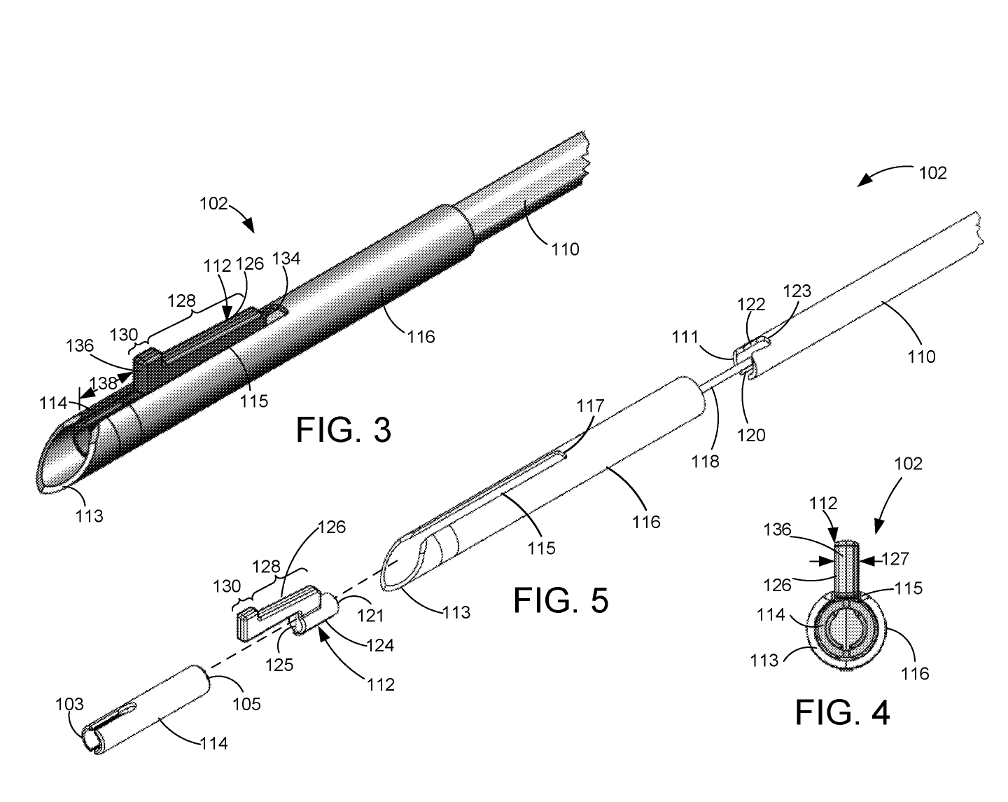

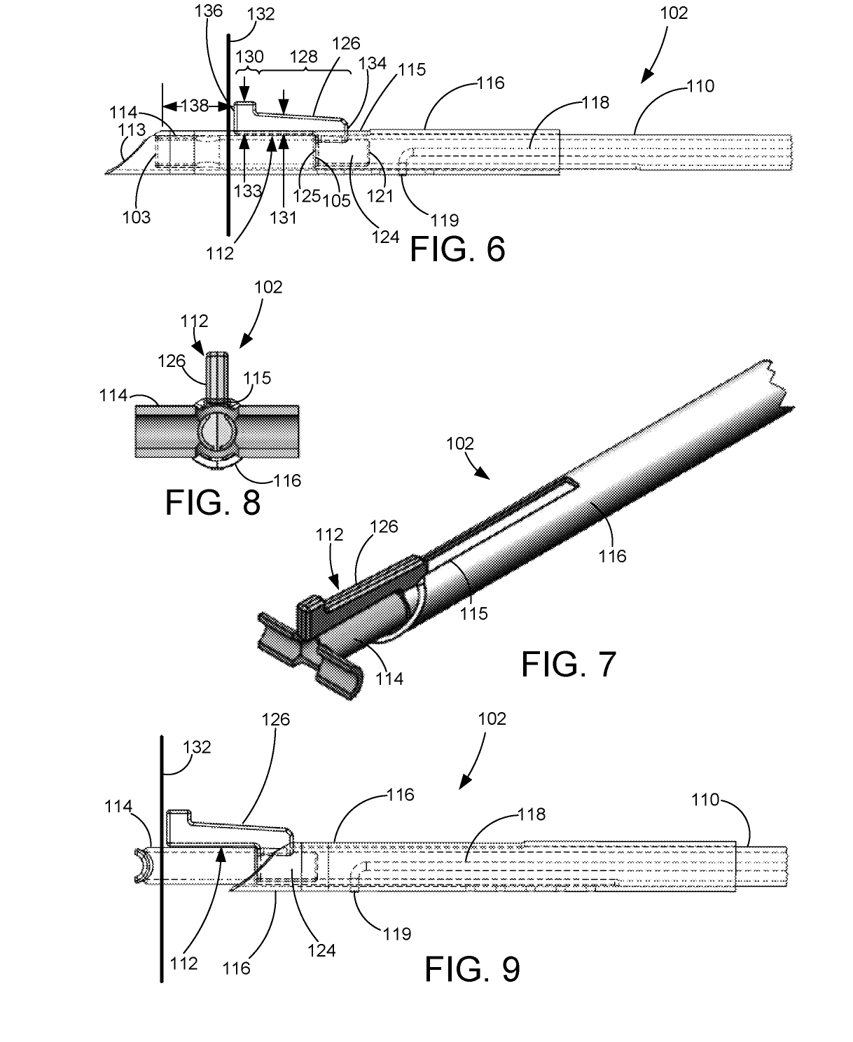

FIG. 3 illustrates an enlarged perspective view of an end portion of nose assembly 102 of FIG. 2 in a loaded configuration according to an embodiment. FIG. 4 illustrates a medial end view of FIG. 3 and FIG. 5 illustrates an exploded perspective view of the end portion of nose assembly 102 of FIG. 3. The illustrated end portion of nose assembly 102 includes an end portion of positioning rod 110, hollow cutting sheath 116, an end portion of actuation member 118, a visual indicator 112 and a ventilation tube 114. Positioning rod 110 may be made of stainless-steel tubing or other suitable metallic or plastic tubing and includes a first slot 120 that extends from a distal end 111 to a terminating point (not illustrated) and an optional second slot 122 that extends from distal end 111 to a terminating point 123 and is located opposite from first slot 120. Hollow cutting sheath 116 may be made of thin-walled stainless-steel tubing. However, other thin-walled metallic or plastic tubing may also be suitable as long as the material provides sufficient rigidity to constrain, compress or otherwise deform ventilation tube 114 before deployment into a body. Hollow cutting sheath 116 includes a distal cutting edge 113, which, for example, may be a beveled cutting edge, and a cutting sheath slot 115 that extends from distal cutting edge 113 to terminating end 117. Actuation member 118 may be made of a flexible material, such as but not limited to plastic or thin metal wire and runs from pull mechanism 106 (FIG. 2) and extends through and/or down the inside of positioning rod 110 and is fixedly attached to cutting sheath 116. Actuation member 118 passes through first slot 120 in positioning rod 110 to couple to or attach to an interior wall of hollow cutting sheath 116. The end portion of actuation member 118 is shown more clearly in FIG. 6. Optional second slot 122 may be, but not necessarily, provided as a feature for receiving and securing a visual indicator.

Visual indicator 112 mates with and is coupled to distal end 111 of positioning rod 110. Visual indicator 112 may be rigid, semi-rigid or flexible and made of, for example, plastic, a thermoplastic polymer, such as PTFE, silicone, rubber, etc. and may be adhered to, molded to or otherwise engaged with positioning rod 110. Under one embodiment, visual indicator 112 may be coupled to or engaged with distal end 111 of positioning rod 110 without the use of adhesives, which will be discussed below. Visual indicator 112 includes a base 124 and a tab 126 that protrudes from base 124. Tab 126 has an elongated portion 128 and a fin portion 130. While elongated portion 128 and fin portion 130 have a uniform width, fin portion 130 has a height that is greater than the height of elongated portion 128. Ventilation tube 114 has a medial end 103 and a lateral end 105 and includes a material that allows the tube to remain in a deformed or compressed state inside cutting sheath 116 before deployment into a body. Medial end 103 is configured to be positioned on a medial side of a membrane of the body or internal to the membrane while lateral end 105 is configured to be positioned on a lateral side of the membrane or external to the membrane. After insertion through a target membrane, the tube is allowed to re-form, decompress, unfold, and/or deploy and anchor in place.

FIG. 6 illustrates a side view of FIG. 3 including further illustrating a position of nose assembly 102 after incising a tympanic membrane 132 but before tube deployment according to an embodiment. In the configuration illustrated in FIGS. 3-6, hollow cutting sheath 116 at least partially surrounds distal end 111 of positioning rod 110 and at least partially surrounds visual indicator 112. In addition, hollow cutting sheath 116 houses or is loaded with ventilation tube 114, which is in a deformed state. Base 124 of visual indicator 112 includes a first end 121 mated, engaged or coupled to distal end 111 of positioning rod 110 and a second end 125 positioned inside hollow cutting sheath 116 adjacent to lateral end 105 of ventilation tube 114. Tab 126 of visual indicator 112 protrudes from base 124 through cutting sheath slot 115 and outwardly from an outer surface of cutting sheath 116. Therefore at least a width of elongated portion 128 of tab 126 is sized to fit through a width of cutting sheath slot 115.

Tab 126 includes a first end 134 and a second end 136. Elongated portion 128 of tab 126 extends distally from first end 134 along the cutting sheath slot 115 and terminates at fin portion 130. Fin portion 130 extends distally from elongated portion 128 and terminates at second end 136. As illustrated in FIGS. 3-6, tab 126 includes the same width 127 (FIG. 4) along the entirety of tab 126 including the same width 127 along elongated portion 128 and along fin portion 130. Fin portion 130, however, has a height 133 (FIG. 6) that is greater than a height 131 (FIG. 6) of elongated portion 128. In addition, before tube deployment, second end 136 is positioned at a spaced distance 138 from distal cutting edge 113 that is equal to a depth of the incision to be made in membrane 132 of a patient. Therefore, when in a loaded configuration as illustrated in FIGS. 3-6 and a clinician is piercing and making an incision in membrane 132 of a patient using distal cutting edge 113, the clinician manually advances or inserts cutting sheath 116 only up to second end 136 of tab 126 as illustrated in FIG. 6 so that tab 126 of visual indicator 112 remains viewable to the clinician and proper incision depth is maintained.

The embodiment illustrated in FIGS. 3-6 shows tab 126 of visual indicator 112 including fin 130 that has a stepped-up body and has a width that is the same width as elongated portion 128. This means that fin 130 provides visualization of the target membrane past fin 130 without obstruction by elongated portion 128 during insertion and a view of the end of visual indicator 112 when near the incised membrane after insertion. While it is possible to over insert fin 130, the width of fin 130 is such that minimal damage to the membrane would occur when over inserted.

FIG. 7 illustrates a perspective view of the end portion of nose assembly 102 of FIG. 3 in a deployed configuration according to an embodiment. FIG. 8 illustrates a medial end view of FIG. 7, and FIG. 9 illustrates a side view of FIG. 7 including further illustrating a position of nose assembly 102 after ventilation tube 114 is deployed and anchored in tympanic membrane 132. To deploy and anchor ventilation tube 114 in tympanic membrane 132, pull mechanism 106 (FIG. 2) actuates actuation member 118 to withdraw or retract hollow cutting sheath 116 from membrane 132 of the patient and from around ventilation tube 114. While pull mechanism 106 pulls actuation member 118, a distal end 119 (FIGS. 6 and 9) of actuation member 118, which is coupled to hollow cutting sheath 116, moves within first slot 120 in the positioning rod 110 and prevents the cutting sheath 116 from rotating about its axis, thereby keeping the constrained ventilation tube 114 in a predictable alignment during insertion. In addition, first end 134 of visual indicator 112 moves along the cutting sheath slot 115 and also prevents cutting sheath 116 from rotating about its axis, thereby also keeping the constrained ventilation tube 114 in a predictable alignment during insertion. The retraction of hollow cutting sheath 116 from around ventilation tube 114 causes ventilation tube 114 to re-form, unfold and anchor in membrane 132 while positioning rod 110 holds ventilation tube 114 in place in membrane 132. For example, as illustrated in the figures, ventilation tube 114 may be a T-tube, which may have a pair of flanges. While loaded in cutting sheath 116, the flanges of tube 114 are compressed or folded inwardly. After ventilation tube 114 is deployed and the flanges of tube 114 unfold outwardly, nose assembly 102 including cutting sheath 116, visual indicator 112 and positioning rod 110 are completely withdrawn from the patient’s body.

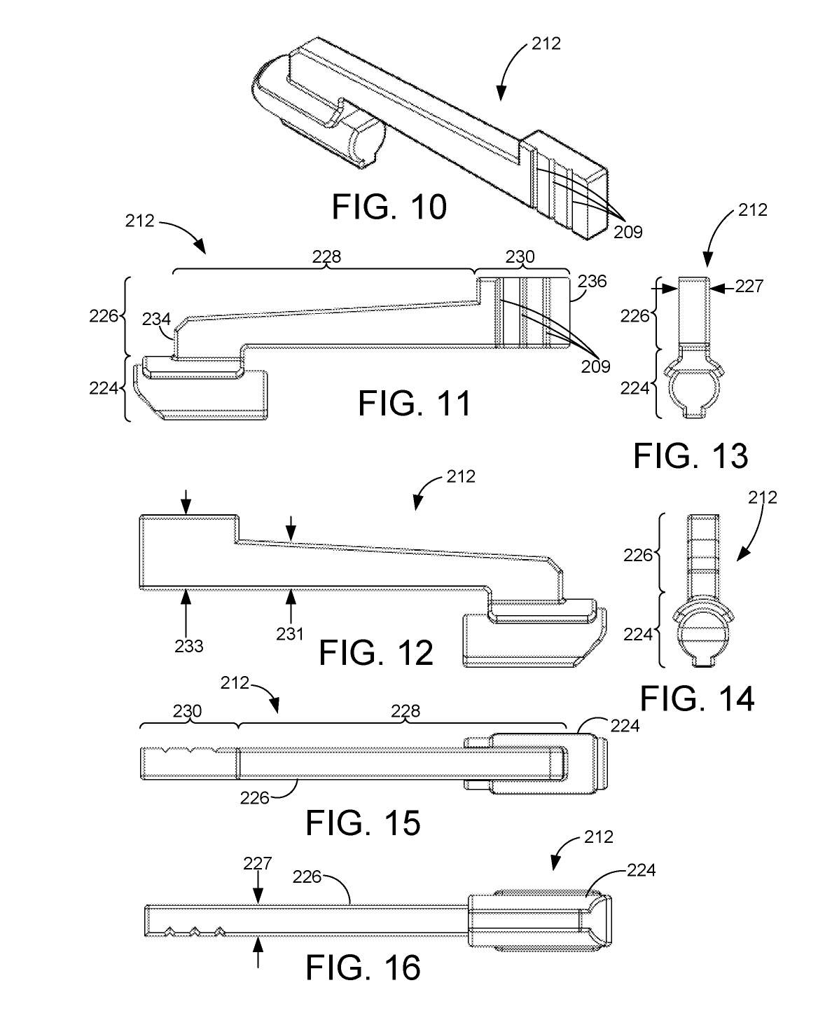

FIG. 10 illustrates a perspective view of a visual indicator 212 according to another embodiment. FIG. 11 is a right side view, FIG. 12 is a left side view, FIG. 13 is a medial end view, FIG. 14 is a lateral end view, FIG. 15 is a top view and FIG. 16 is a bottom view of visual indicator 212. Similar to visual indicator 112, visual indicator 212 may be rigid, semi-rigid or flexible and made of, for example, plastic, a thermoplastic polymer, such as PTFE, silicone, such as 70-80 durometer silicone, rubber, etc. and is engaged with a positioning rod described below. In this embodiment, visual indicator 212 is configured to engage with a positioning rod without the use of adhesives. Visual indicator 212 includes a base 224 and a tab 226 that protrudes from base 224. Tab 226 has an elongated portion 228 and a fin portion 230. Tab 226 includes a first end 234 and a second end 236. Elongated portion 228 of tab 226 extends distally from first end 234 and terminates at fin portion 230. Fin portion 230 extends distally from elongated portion 228 and terminates at second end 236. As illustrated in FIGS. 10-16, tab 226 includes the same width 227 along the entirety of tab 226 including the same width 227 along elongated portion 228 and along fin portion 230. Fin portion 230, however, has a height 233 that is greater than a variable height 231 of elongated portion 228.

In addition, fin portion 230 of tab 226 includes a plurality of spaced apart grooves 209 that extend along height 233 of fin portion 230. The plurality of grooves are each substantially parallel to each other and are indicative of trimmable portions of the fin portion 230 to adjust a length of tab 226 from first end 234 to second end 236. In particular, if the groove closest to second end 236 is trimmed, the length of tab 226 decreases. But if the middle groove or the groove closest to elongated portion 228 is trimmed the length of 226 further decreases.

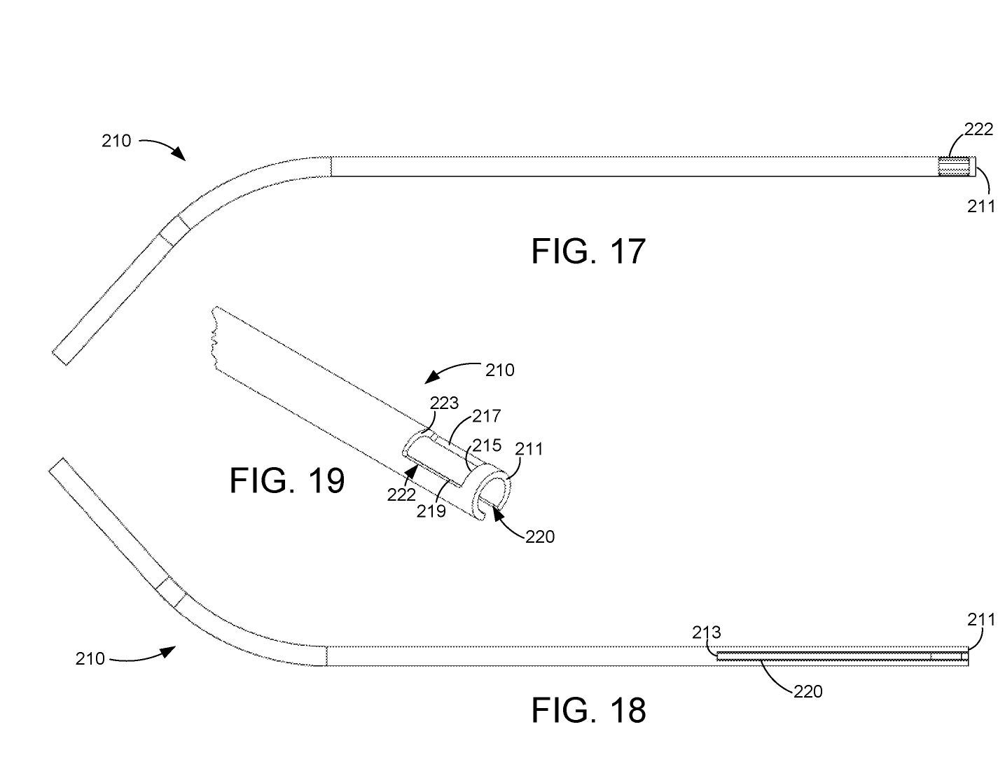

FIG. 17 is a top view and FIG. 18 is a bottom view of another embodiment of a positioning rod 210 that may be used in FIG. 2 tube deploying system 100. FIG. 19 is an enlarged perspective view of a distal end 211 of positioning rod 210 according to another embodiment. Positioning rod 210 may be made of stainless-steel tubing or other suitable metallic or plastic tubing and includes a first slot 220 that extends from distal end 211 to a terminating point 213 and a second slot 222 that is defined between a first end 215 located a spaced distance from distal end 211 of positioning rod 210 and an opposing second end 223 and between a first lateral side 217 and an opposing second lateral side 219. Second slot 222 is located opposite from first slot 220.

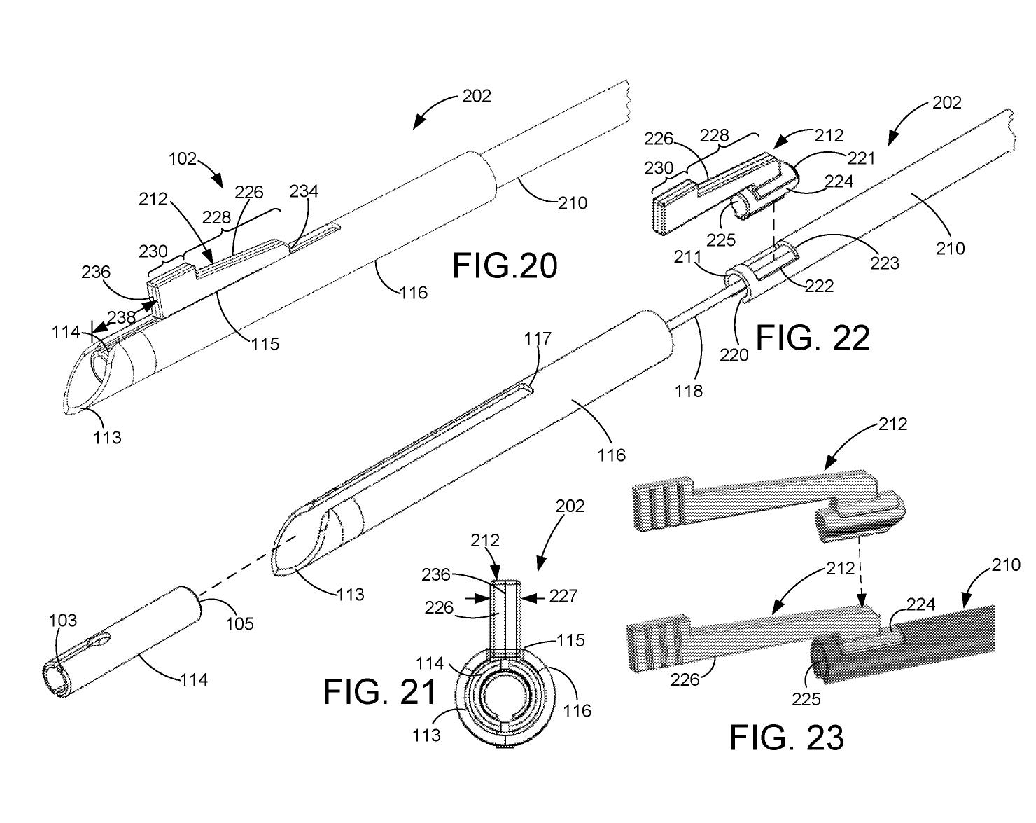

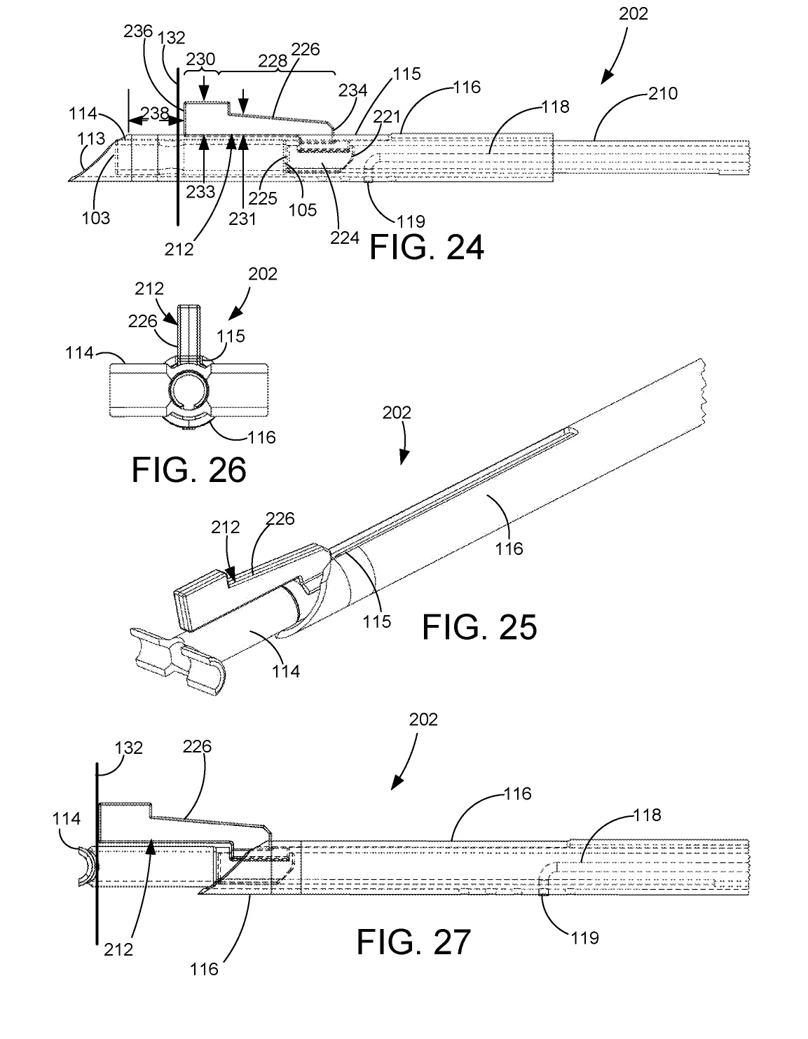

FIG. 20 illustrates an enlarged perspective view of an end portion of another embodiment of a nose assembly 202 in a loaded configuration that may be used in the FIG. 2 tube deploying system 100. FIG. 21 illustrates a medial end view of FIG. 20, FIG. 22 illustrates an exploded perspective view of the end portion of nose assembly 202 of FIG. 20 and FIG. 23 illustrates an enlarged perspective view of distal end 211 of positioning rod 210 with visual indicator 212 assembled thereto and all other components deleted from view. The illustrated end portion of nose assembly 202 includes an end portion of positioning rod 210 (illustrated in FIGS. 17-19), hollow cutting sheath 116, an end portion of actuation member 118, visual indicator 212 (FIGS. 10-16) and ventilation tube 114. Similar to FIGS. 3-9, actuation member 118 passes through first slot 220 in positioning rod 210 to couple to or attach to an interior wall of hollow cutting sheath 116. The end portion of actuation member 118 is shown more clearly in FIG. 24. As illustrated in FIGS. 22 and 23, base 224 of visual indicator 212 is configured to snap-fit into second slot 222 of positioning rod 211. In particular, base 224 is designed to snap-fit internally through second slot 222 and sit inside positioning rod 210 and adjacent to distal end 211.

It should be understood that to engage visual indicator 212 with positioning rod 210, visual indicator 212 should be engaged while positioning rod 210 is assembled with actuation member 118 and cutting sheath 116, but before ventilation tube 114 is loaded into the cutting sheath. In particular, actuation member 118 is coupled internally to cutting sheath 116 and through first slot 220 in positioning rod 210. Using actuation member 118, cutting edge 113 of cutting sheath 116 is retracted past second slot 222 of positioning rod 210. Visual indicator 212 is engaged or snap-fit through second slot 222 and sits securely within positioning rod 210 adjacent distal end 211. Actuation member 118 then slides cutting sheath 116 back over visual indicator 212 so that tab 226 extends outwardly through cutting sheath slot 115 to further secure visual indicator 212.

FIG. 24 illustrates a side view of FIG. 20 including further illustrating a position of nose assembly 202 after incising a tympanic membrane 132 but before tube deployment according to an embodiment. In the configuration illustrated in FIGS. 20-24, hollow cutting sheath 116 at least partially surrounds distal end 211 of positioning rod 210 and at least partially surrounds visual indicator 212. In addition, hollow cutting sheath 116 houses or is loaded with ventilation tube 114, which is in a deformed state. Base 224 of visual indicator 212 is located inside positioning rod 210 and includes a first end 221 and a second end 225 with second end 225 being adjacent to distal end 211 of positioning rod 210. In FIG. 24, base 224 is also located inside hollow cutting sheath 116 with lateral end 105 of ventilation tube 114 located adjacent to distal end 211 of positioning rod 210. Tab 226 of visual indicator 212 protrudes from base 224 through second slot 222 of positioning rod 210 and through cutting sheath slot 115 and outwardly from an outer surface of cutting sheath 116. Therefore, a portion of a width of tab 226 is sized to fit through a width of cutting sheath slot 115.

Tab 226 includes a first end 234 and a second end 236. Elongated portion 228 of tab 226 extends distally from first end 234 along the cutting sheath slot 115 and terminates at fin portion 230. Fin portion 230 extends distally from elongated portion 228 and terminates at second end 236. As illustrated in FIGS. 20-23, tab 226 includes the same width 227 (FIGS. 13 and 21) along the entirety of tab 226 including the same width 227 along elongated portion 228 and along fin portion 230. Fin portion 230, however, has a height 233 (FIG. 12) that is greater than a height 231 (FIG. 12) of elongated portion 228. In addition, before tube deployment, second end 236 is positioned at a spaced distance 238 from distal cutting edge 113 that is equal to a depth of the incision to be made in membrane 132 of a patient. Therefore, when in a loaded configuration as illustrated in FIGS. 20-24 and a clinician is piercing and making an incision in membrane 132 of a patient using distal cutting edge 113, the clinician manually advances or inserts cutting sheath 116 only up to second end 236 of tab 226 as illustrated in FIG. 24 so that tab 226 of visual indicator 212 remains viewable to the clinician and proper incision depth is maintained.

The embodiment illustrated in FIGS. 20-24 shows tab 226 of visual indicator 212 including fin 230 that has a stepped-up body and has a width that is the same width as elongated portion 228. This means that fin 230 provides visualization of the target membrane past fin 230 without obstruction by elongated portion 228 during insertion and a view of the end of visual indicator 212 when near the incised membrane after insertion. While it is possible to over insert fin 230, the width of fin 230 is such that minimal damage to the membrane would occur when over inserted.

FIG. 25 illustrates a perspective view of the end portion of nose assembly 202 of FIG. 20 in a deployed configuration according to an embodiment. FIG. 26 illustrates a medial end view of FIG. 25, and FIG. 27 illustrates a side view of FIG. 25 including further illustrating a position of nose assembly 202 after ventilation tube 114 is deployed and anchored in tympanic membrane 132. To deploy and anchor ventilation tube 114 in tympanic membrane 132, pull mechanism 106 (FIG. 2) actuates actuation member 118 to withdraw or retract hollow cutting sheath 116 from membrane 132 of the patient and from around ventilation tube 114. While pull mechanism 106 pulls actuation member 118, a distal end 119 (FIGS. 6 and 9) of actuation member 118, which is coupled to hollow cutting sheath 116, moves within first slot 220 in the positioning rod 210 (see FIG. 18) and prevents the cutting sheath 116 from rotating about its axis, thereby keeping the constrained ventilation tube 114 in a predictable alignment during insertion. In addition, the cutting sheath slot 115 moves along tab 226 of visual indicator 212 and also prevents cutting sheath 116 from rotating about its axis, thereby also keeping the constrained ventilation tube 114 in a predictable alignment during insertion. The retraction of hollow cutting sheath 116 from around ventilation tube 114 causes ventilation tube 114 to re-form, unfold and anchor in membrane 132 while positioning rod 210 holds ventilation tube 114 in place in membrane 132. For example, as illustrated in the figures, ventilation tube 114 may be a T-tube, which may have a pair of flanges. While loaded in cutting sheath 116, the flanges of tube 114 are compressed or folded inwardly. After ventilation tube 114 is deployed and the flanges of tube 114 unfold outwardly, nose assembly 202 including cutting sheath 116, visual indicator 12 and positioning rod 210 are completely withdrawn from the patient’s body.

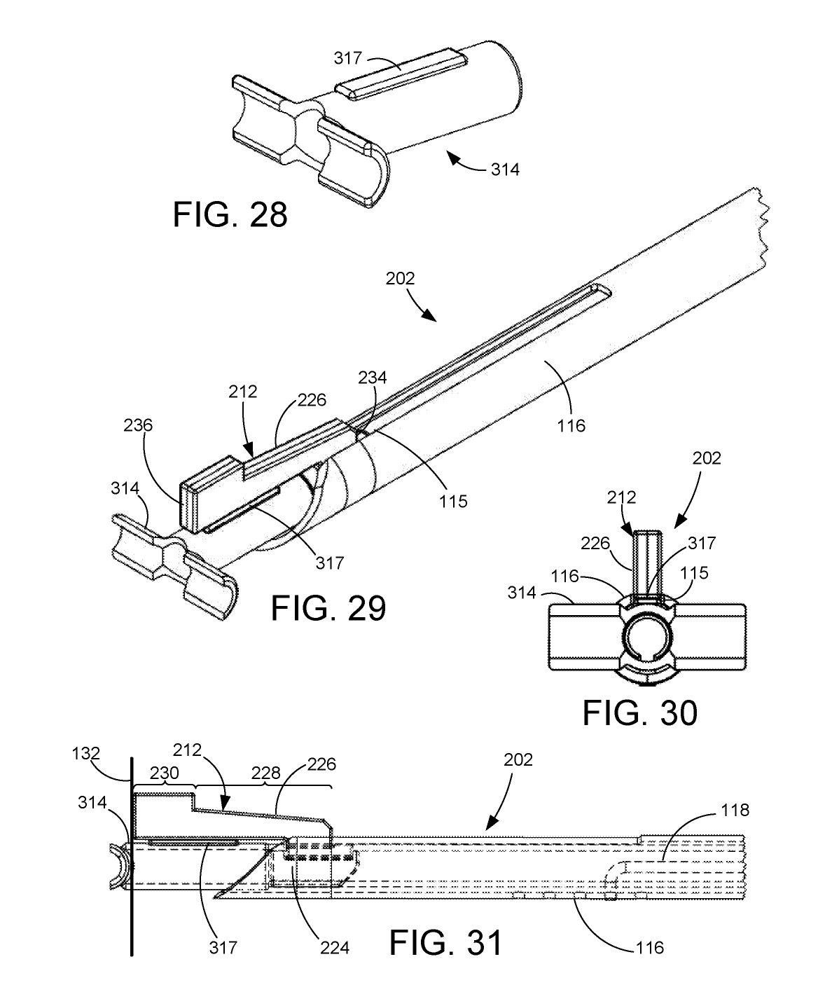

FIG. 28 illustrates a perspective view of a ventilation tube 314 according to another embodiment. Ventilation tube 314 is similar to the T-tube of ventilation tube 114, however, ventilation tube 314 includes an alignment feature 317. Alignment feature 317 may be a single body as illustrated in FIG. 28 or may be a series of in-line bodies. Regardless, alignment feature 317 is integral with and protrudes from the top of ventilation tube 314 and oriented to be raised from tube 314 in a plane that is substantially perpendicular to the plane to which the flanges of the T-tube 314 unfold. Ventilation tube 314 is loaded in the end portion of nose assembly 202 like ventilation tube 114 in FIG. 24 in undeployed state.

FIG. 29 illustrates a perspective view of the end portion of nose assembly 202 after tube deployment according to an embodiment, FIG. 30 illustrates a medial end view of FIG. 25 and FIG. 31 illustrates a side view of FIG. 29 with ventilation tube 314 of FIG. 28 anchored in tympanic membrane 132. As illustrated, slot 115 of hollow cutting sheath 116 slides along alignment feature 317 of ventilation tube 314 and ensures the orientation of ventilation tube 314 before and while the tube is being deployed.

In an undeployed state, hollow cutting sheath 116 houses ventilation tube 314 in a deformed state. Base 224 of visual indicator 212 engages with positioning rod 210 and is located inside an interior of positioning rod 210 and hollow cutting sheath 116. Tab 226 of visual indicator 212 protrudes from base 224 through second slot 222 of positioning rod 210 and through cutting sheath slot 115, outwardly from an outer surface of cutting sheath 116 and extends distally from first end 234 to second end 236. A bottom of tab 226 sits on alignment feature 317. To deploy and anchor ventilation tube 314 in membrane 132, pull mechanism 106 (FIG. 2) actuates actuation member 118 to withdraw or retract hollow cutting sheath 116 from membrane 132 and from around ventilation tube 314, which means a bottom of tab 226 including a portion of elongated portion 228 and fin portion 230 remain seated on alignment feature 317 as hollow cutting sheath 116 is withdrawn or retracted from around ventilation tube 314 and ventilation tube 314 re-forms, unfolds and anchors in membrane 132 while visual indicator 212 and positioning rod 210 hold ventilation tube 314 in place. After ventilation tube 314 is deployed and anchored, nose assembly 202 including visual indicator 212 and positioning rod 210 is completely withdrawn from the patient’s ear.

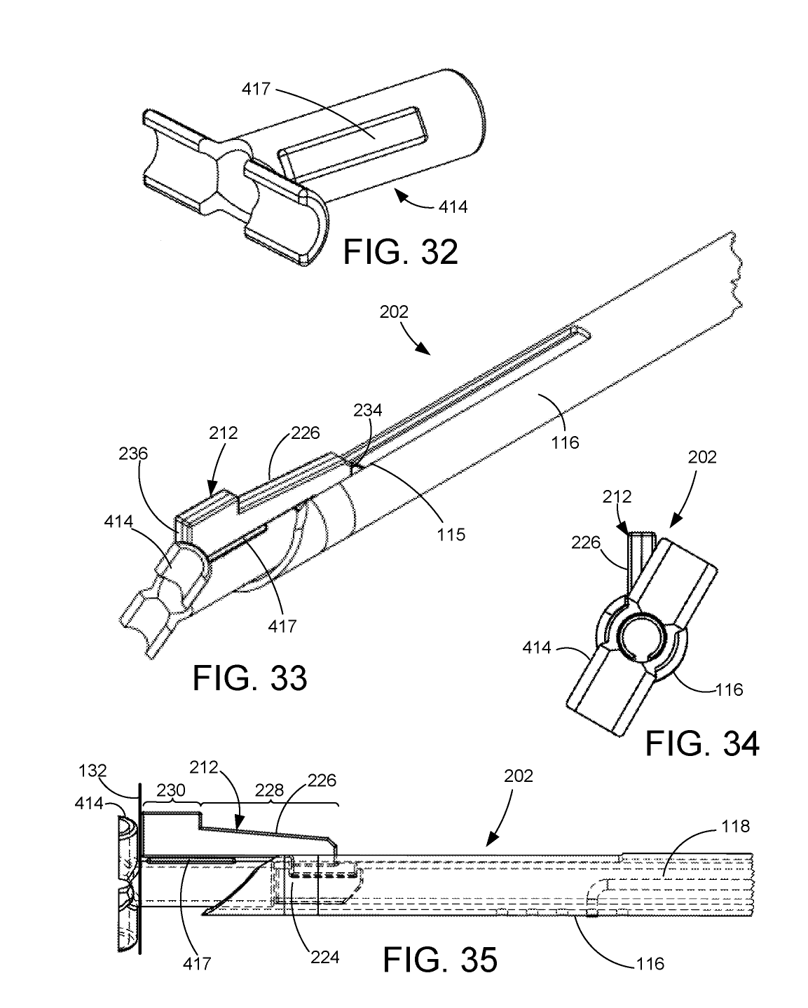

FIG. 32 illustrates a perspective view of a ventilation tube 414 according to another embodiment. Ventilation tube 414 is similar to the T-tube of ventilation tube 114, however, ventilation tube 414 includes an alignment feature 417. Alignment feature 417 may be a single body as illustrated in FIG. 32 or may be a series of in-line bodies. Regardless, alignment feature 417 is integral with and protrudes from a side of ventilation tube 414 and is oriented to be raised from tube 414 in a plane that is different from the plane to which alignment feature 317 of ventilation tube 314 is oriented. As illustrated, alignment feature 417 is oriented and protrudes from tube 414 in a plane that is at an angle that is 90 degrees or less from the plane to which the flanges of T-tube 414 unfold.

FIG. 33 illustrates a perspective view of the end portion of nose assembly 202 after tube deployment according to an embodiment, FIG. 34 illustrates a medial end view of FIG. 33 and FIG. 19 illustrates a side view of FIG. 33 with the ventilation tube of FIG. 32 anchored in the tympanic membrane. As illustrated, slot 115 of hollow cutting sheath 116 slides along alignment feature 417 of ventilation tube 414 and ensures the orientation of ventilation tube 414 before and while the tube is being deployed.

In an undeployed state, hollow cutting sheath 116 houses ventilation tube 414 in a deformed state. Base 224 of visual indicator 212 engages with positioning rod 210 and is located inside an interior of positioning rod 210 and hollow cutting sheath 116. Tab 226 of visual indicator 212 protrudes from base 224 through second slot 222 of positioning rod 210 and through cutting sheath slot 115, outwardly from an outer surface of cutting sheath 116 and extends distally from first end 234 to second end 236. A bottom of tab 1226 sits on alignment feature 417. To deploy and anchor ventilation tube 414 in membrane 132, pull mechanism 106 (FIG. 2) actuates actuation member 118 to withdraw or retract hollow cutting sheath 116 from membrane 132 and from around ventilation tube 414, which means a bottom of tab 226 including a portion of elongated portion 228 and fin portion 230 remain seated on alignment feature 417 as hollow cutting sheath 116 is withdrawn or retracted from around ventilation tube 414 and ventilation tube 414 re-forms, unfolds and anchors in membrane 132 while visual indicator 412 and positioning rod 410 hold ventilation tube 414 in place. After ventilation tube 414 is deployed and anchored, nose assembly 202 including visual indicator 212 and positioning rod 210 is completely withdrawn from the patient’s ear. As illustrated, alignment feature 417 allows ventilation tube 414 to be placed in a different orientation than ventilation tube 314.

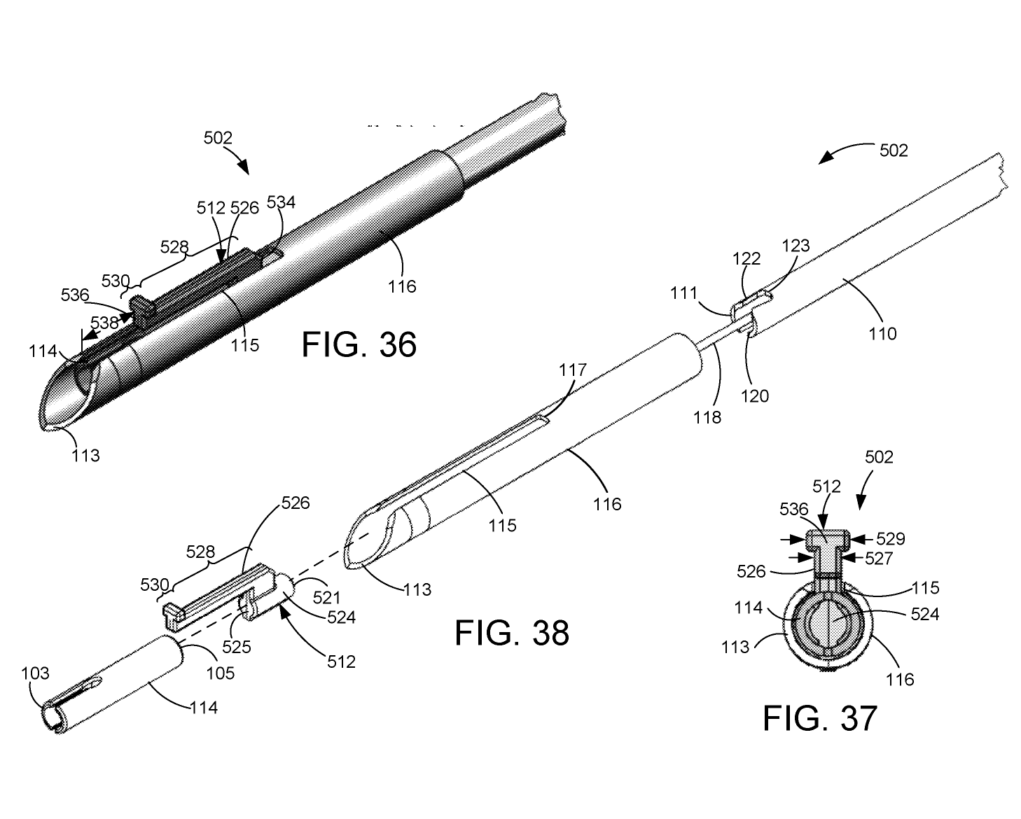

FIG. 36 illustrates a perspective view of an end portion of a nose assembly 502 of a tube deploying system according to an embodiment. FIG. 37 illustrates a medial end view of the end portion of nose assembly 502 of FIG. 36 and FIG. 38 illustrates an exploded perspective view of FIG. 36. The illustrated end portion of nose assembly 502 includes an end portion of positioning rod 110, hollow cutting sheath 116, an end portion of actuation member 118, a visual indicator 512 and ventilation tube 114. Like visual indicator 112, visual indicator 512 mates or engages with distal end 111 of positioning rod 110 and includes a base 524 and a tab 4526 that protrudes from base 524. Tab 526 has an elongated portion 528 and a fin portion 530.

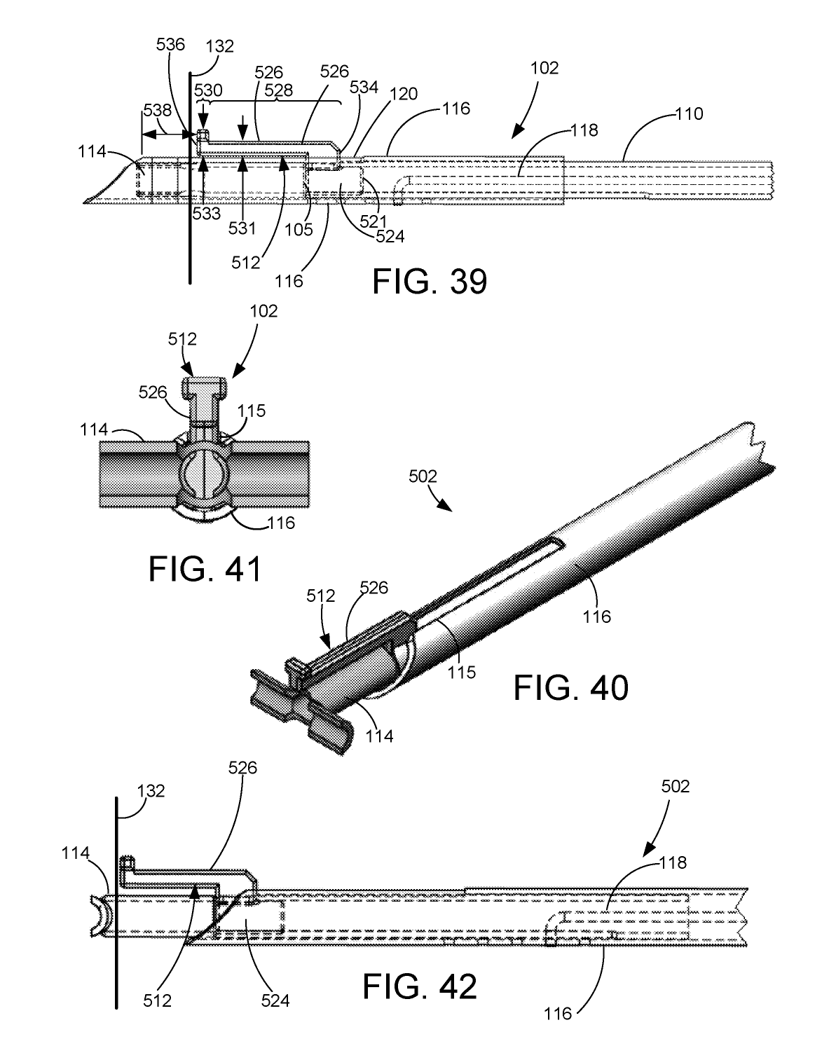

FIG. 39 illustrates a side view of FIG. 36 including further illustrating a position of nose assembly 502 after incising tympanic membrane 132 but before tube deployment and as previously illustrated in FIG. 6 but with a different visual indicator 512. Base 524 of visual indicator 512 includes a first end 521 mated or coupled to distal end 111 of positioning rod 110 and a second end 525 positioned inside hollow cutting sheath 116 adjacent to lateral end 105 of ventilation tube 114. Tab 526 of visual indicator 512 protrudes from base 524 through cutting sheath slot 115 and outwardly from an outer surface of cutting sheath 116. Therefore at least a width of the elongated portion 528 is small enough to fit through a width of cutting sheath slot 115.

Tab 526 includes a first end 534 and a second end 4536. Elongated portion 528 of tab 526 extends distally from first end 534 along the cutting sheath slot 115 and terminates at fin portion 530. Fin portion 530 extends from elongated portion 528 and terminates at second end 536. As illustrated in FIGS. 36-39, elongated portion 528 includes a width 527, while fin portion 530 includes a different width 529. Width 529 is greater than width 527. In addition, height 533 of fin portion 530 is greater than height 531 of elongated portion 528. Width 529 being greater than width 528 and height 533 being greater than height 531 provides improved visibility of the target membrane compared to visual indicator 112. In particular, fin portion 530 having a width 529 that is greater than width 527 of elongated portion 528 provides more areas of visualization that are not obstructed by elongated portion 528 than in visual indicator 112. In addition, a wider fin width provides more haptic feedback to the clinician and resistance to over insertion. Second end 436 is positioned at a space distance 538 from distal cutting edge 113 that is equal to a depth of the incision to be made in membrane 132 of a patient. Therefore, when in a loaded configuration as illustrated in FIGS. 36-39 and a clinician is piercing and making an incision in membrane 132 of a patient using distal cutting edge 113, the clinician manually advances or inserts cutting sheath 116 only up to second end 536 of tab 526 as illustrated in FIG. 39 so that tab 526 of visual indicator 512 remains visible to the clinician and proper incision depth is maintained.

FIG. 40 illustrates a perspective view of the end portion of nose assembly 502 of FIG. 36 in a tube deployment configuration according to an embodiment. FIG. 41 illustrates a medial end view of FIG. 40, and FIG. 42 illustrates a side view of FIG. 40 including further illustrating a position of nose assembly 502 after ventilation tube 114 is deployed and anchored in tympanic membrane 132. To deploy and anchor ventilation tube 114 in tympanic membrane 132, pull mechanism 106 (FIG. 2) actuates actuation member 118 to withdraw or retract hollow cutting sheath 116 from membrane 132 and from around ventilation tube 114. The retraction of hollow cutting sheath 116 from around ventilation tube 114 causes ventilation tube 114 to re-form, unfold and anchor in membrane 132 while visual indicator 512 and positioning rod 110 hold ventilation tube 114 in place. After ventilation tube 114 is deployed and anchored, nose assembly 502 including visual indicator 512 and positioning rod 110 is completely withdrawn from the patient’s ear.

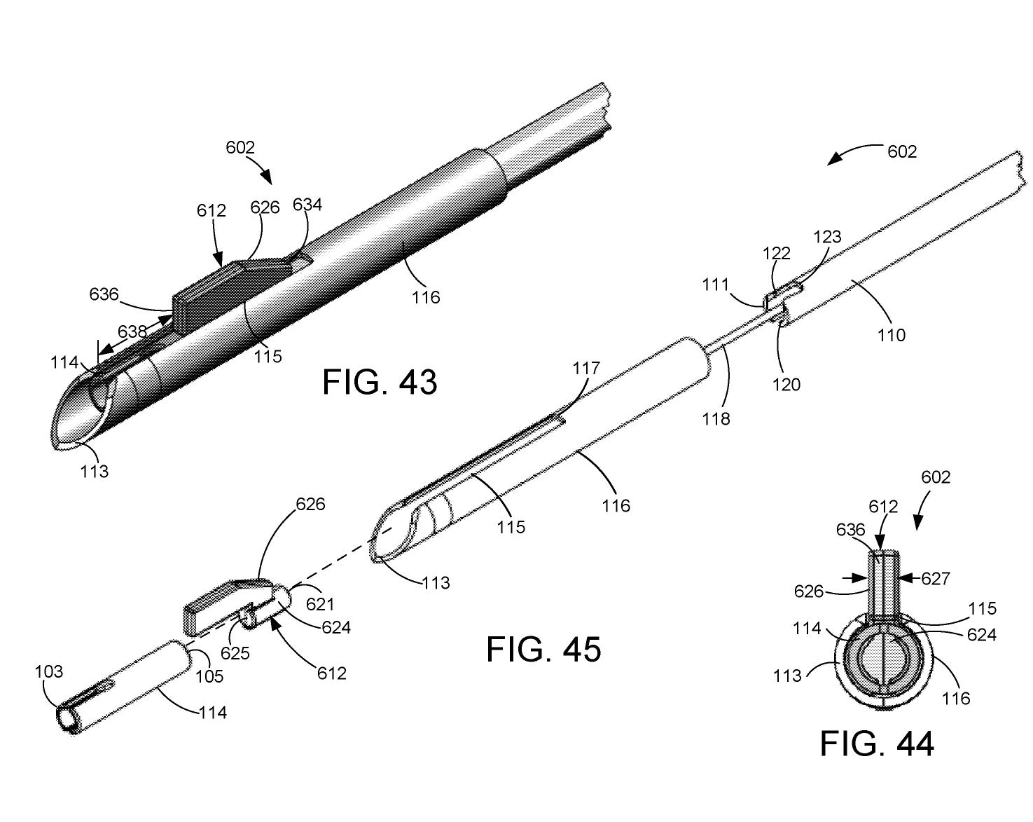

FIG. 43 illustrates a perspective view of an end portion of a nose assembly 602 of a tube deploying system according to an embodiment. FIG. 44 illustrates a medial end view of FIG. 43 and FIG. 45 illustrates an exploded perspective view of the end portion of nose assembly 103 of FIG. 43 in a loaded configuration. The illustrated end portion of nose assembly 602 includes an end portion of positioning rod 110, hollow cutting sheath 116, an end portion of actuation member 118, a visual indicator 612 and ventilation tube 114. Like visual indicator 112, visual indicator 612 mates or engages with and is coupled to distal end 111 of positioning rod 110 and includes a base 624 and a tab 626 that protrudes from base 524.

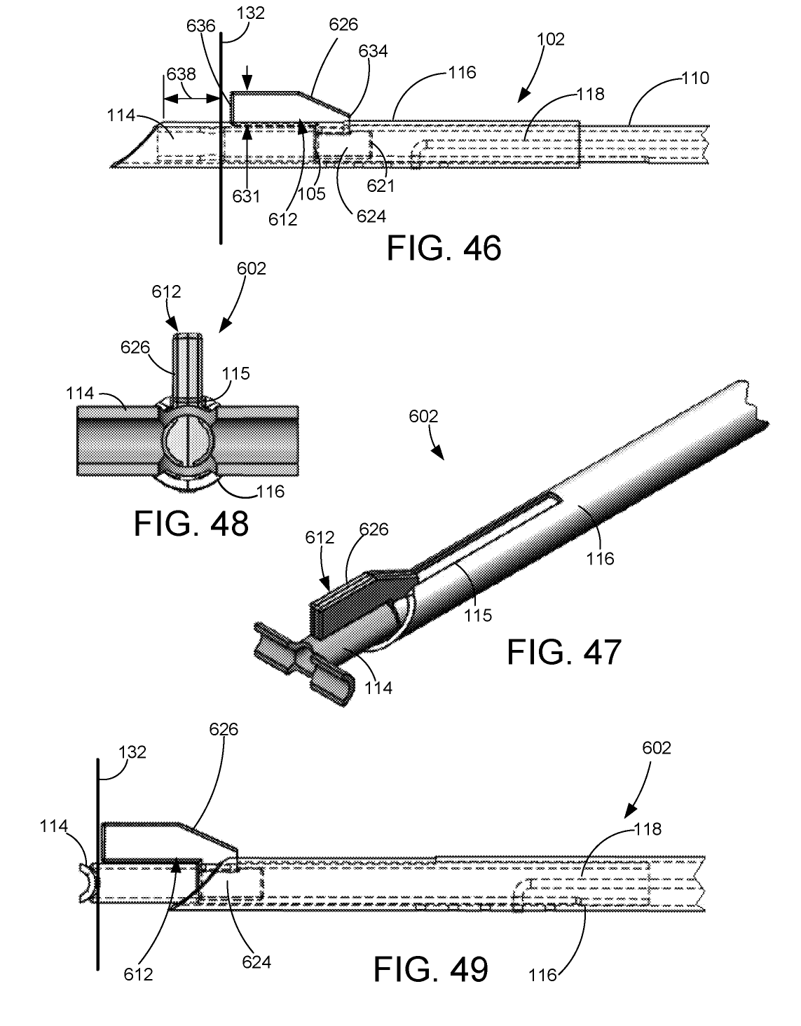

FIG. 46 illustrates a side view of FIG. 43 including further illustrating a position of nose assembly 602 after incising tympanic membrane 132 but before tube deployment and as previously illustrated in FIG. 6 but with a different visual indicator 612. Base 624 of visual indicator 612 includes a first end 621 mated or coupled to distal end 111 of positioning rod 110 and a second end 625 is positioned inside hollow cutting sheath 116 adjacent to lateral end 105 of ventilation tube 114. Tab 626 of visual indicator 612 protrudes from base 624 through cutting sheath slot 115 and outwardly from an outer surface of cutting sheath 116. Therefore, at least the width of a portion of tab 626 is small enough to fit through a width of cutting sheath slot 115.

Tab 626 includes a first end 634 and a second end 636. Tab 626 of visual indicator 612 extends from first end 634 along the cutting sheath slot 115 and terminates at second end 636. As illustrated in FIGS. 43-46, tab 626 includes a width 627. While tab 626 has width 627 that is the same throughout its entirety, height 631 of tab 626 varies. Height 631 of tab 626 at first end 634 is less than height 631 of tab 626 at second end 636. The variable height 631 makes tab 626 more rigid than tabs 126 and 426 and less likely to be over inserted past the target membrane. If, however, an over insertion occurs, the taper to first end 634 will make it easy to withdraw and mitigate damage to the target membrane. Second end 636 is positioned at a spaced distance 638 from cutting edge 113 that is equal to a depth of the incision to be made in membrane 132 of a patient. Therefore, when in a loaded configuration as illustrated in FIGS. 28-30 and a clinician is piercing and making an incision in membrane 132 of a patient using distal cutting edge 113, the clinician manually advances or inserts cutting sheath 116 only up to second end 636 of tab 626 as illustrated in FIG. 30 so that tab 626 of visual indicator 612 remains visible to the clinician and proper incision depth is maintained.

FIG. 47 illustrates a perspective view of the end portion of nose assembly 602 of FIG. 43 in a tube deployment configuration according to an embodiment. FIG. 48 illustrates a medial end view of FIG. 43, and FIG. 49 illustrates a side view of FIG. 43 including further illustrating a position of nose assembly 602 after ventilation tube 114 is deployed and anchored in tympanic membrane 132. To deploy and anchor ventilation tube 114 in membrane 132, pull mechanism 106 (FIG. 2) actuates actuation member 118 to withdraw or retract hollow cutting sheath 116 from membrane 132 and from around ventilation tube 114. The retraction of hollow cutting sheath 116 from around ventilation tube 114 causes ventilation tube 114 to re-form, unfold and anchor in membrane 132 while visual indicator 612 and positioning rod 110 hold ventilation tube 114 in place. After ventilation tube 114 is deployed and anchored, nose assembly 602 including visual indicator 612 and positioning rod 110 is completely withdrawn from the patient’s ear.

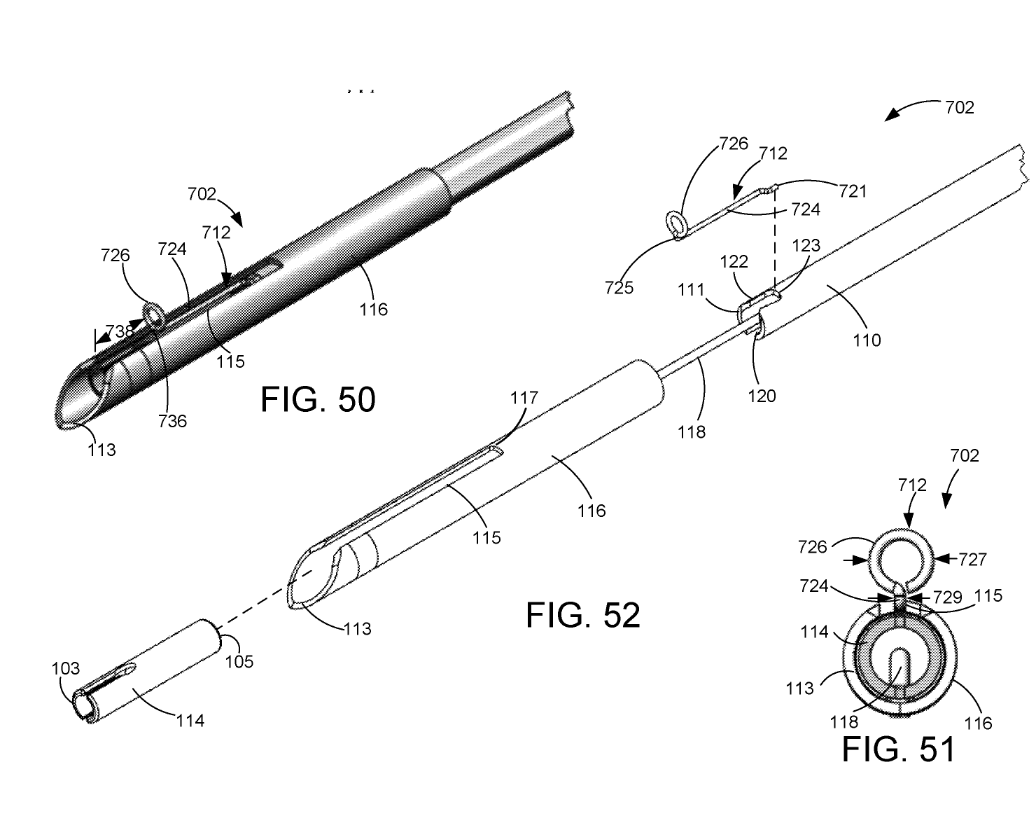

FIG. 50 illustrates a perspective view of an end portion of nose assembly 702 of a tube deploying system according to an embodiment. FIG. 51 illustrates a medial end view of FIG. 50 and FIG. 52 illustrates an exploded perspective view of the end portion of FIG. 50. The illustrated end portion of nose assembly 702 includes an end portion of positioning rod 110, hollow cutting sheath 116, an end portion of actuation member 118, a visual indicator 712 and ventilation tube 114. Visual indicator 712 includes a base 724 that extends from a first end 721 to a second end 725 and a tab 727 that protrudes from the second end 725 of base 724. First end 721 of base 724 is configured to mate with and be coupled to distal end 111 of positioning rod 110. Visual indicator 712 may be rigid, semi-rigid or flexible and made of, for example, metal, plastic, a thermoplastic polymer, such as PTFE, silicone, rubber, etc. and may be soldered, welded adhered, or molded to positioning rod 110.

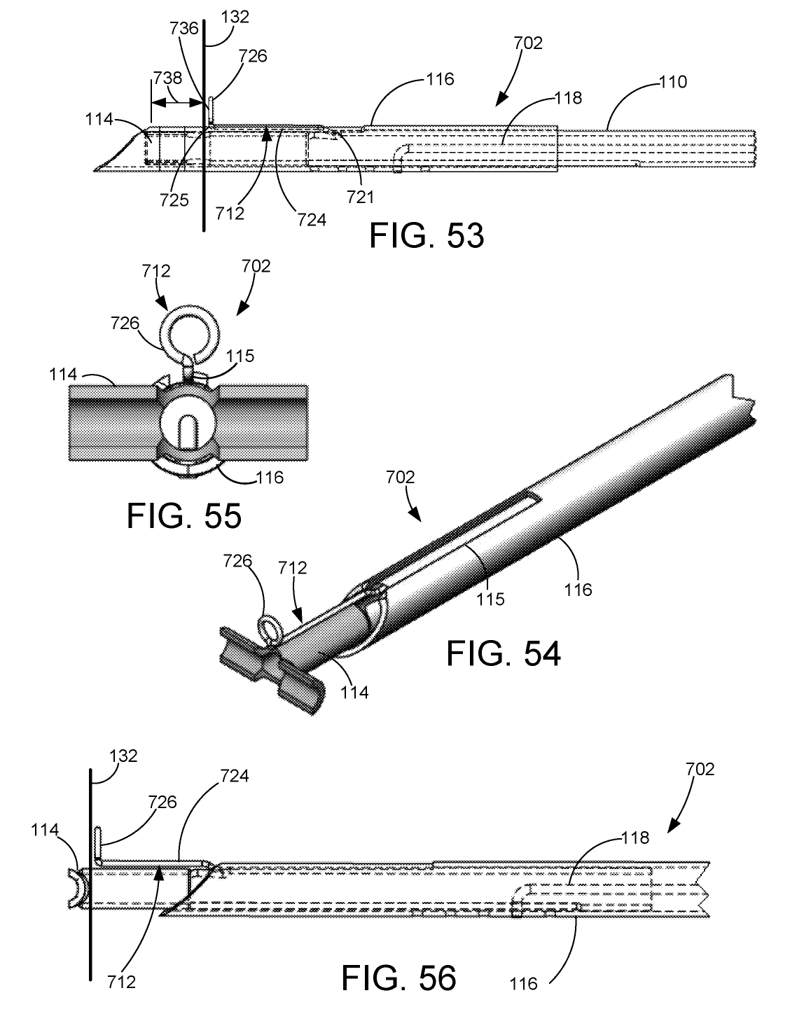

FIG. 53 illustrates a side view of FIG. 50 including further illustrating a position of nose assembly 702 after incising tympanic membrane 132 but before tube deployment and as previously illustrated in FIG. 6 but with a different visual indicator 712. As discussed above, tab 726 of visual indicator 712 protrudes from second end 725 of base 724 through cutting sheath slot 115 and outwardly from the outer surface of cutting sheath 116. Therefore at least a width of a portion of visual indicator 712 is small enough to fit through a width of cutting sheath slot 115.

Base 724 of visual indicator 712 extends distally from first end 721 along the cutting sheath slot 115 and terminates at second end 725 and tab 727 protrudes from second end 625. As illustrated in FIGS. 50-53, tab 726 is a circular shaped ring 736 that includes a width 727 that is greater than a width 729 of base 724. The embodiment of visual indicator 712 illustrated in FIGS. 50-53 is less obstructive of the view of the target membrane than visual indicators 126, 526 and 626 due to the thin shape of ring 736. Second end 725 of base 724 is positioned at a spaced distance 738 from distal cutting edge 113 that is equal to a depth of the incision to be made in target membrane 132 of a patient. Therefore, when in a loaded configuration as illustrated in FIGS. 35-37 and a clinician is piercing and making an incision in target membrane 132 of a patient using distal cutting edge 113, the clinician manually advances or inserts cutting sheath 116 only up to second end 725 or where tab 726 protrudes from base 724 of visual indicator 712 so that tab 726 of visual indicator 712 remains visible to the clinician and proper incision depth is maintained.

FIG. 54 illustrates a perspective view of the end portion of nose assembly 702 of FIG. 50 in a tube deployment configuration according to an embodiment. FIG. 55 illustrates a medial end view of FIG. 54, and FIG. 56 illustrates a side view of FIG. 55 including further illustrating a position of nose assembly 702 after ventilation tube 114 is deployed and anchored in tympanic membrane 132. To deploy and anchor ventilation tube 114 in tympanic membrane 132, pull mechanism 106 (FIG. 2) actuates actuation member 118 to withdraw or retract hollow cutting sheath 116 from membrane 132 and from around ventilation tube 114. The retraction of hollow cutting sheath 116 from around ventilation tube 114 causes ventilation tube 114 to re-form, unfold and anchor in membrane 132 while visual indicator 712 and positioning rod 110 hold ventilation tube 114 in place. After ventilation tube 114 is deployed and anchored, nose assembly 702 including visual indicator 712 and positioning rod 110 is completely withdrawn from the patient’s ear.

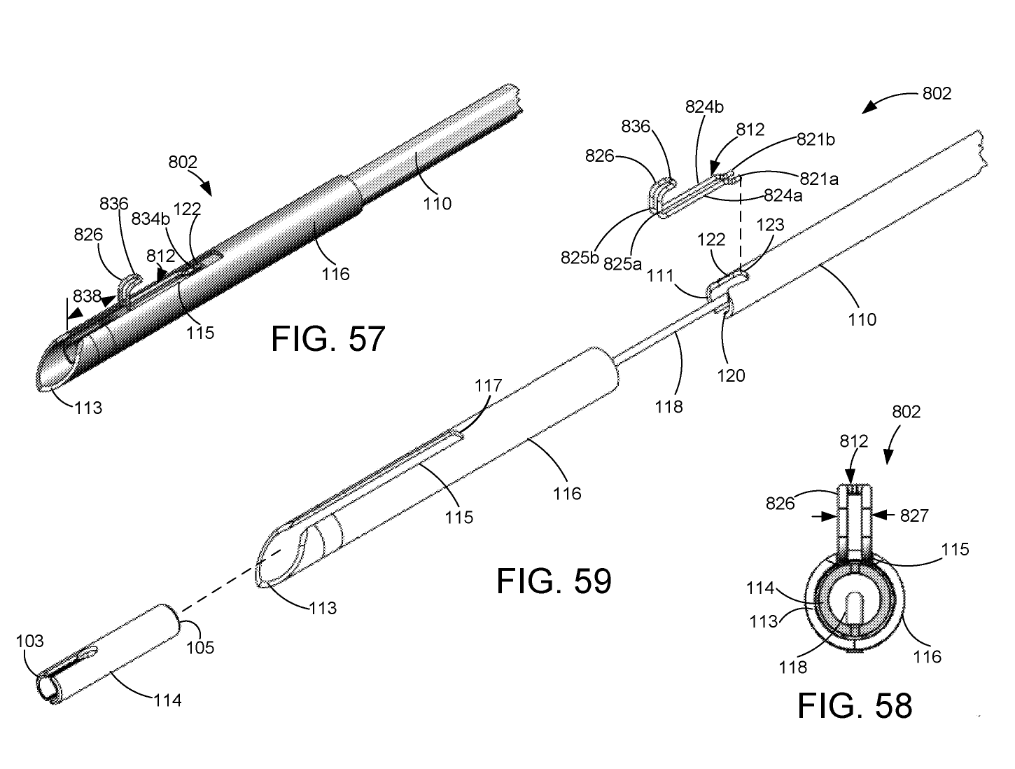

FIG. 57 illustrates a perspective view of an end portion of nose assembly 802 of a tube deploying system according to an embodiment. FIG. 58 illustrates a medial end view of FIG. 57 and FIG. 59 illustrates an exploded perspective view of the end portion of nose assembly 102 of FIG. 57 and The end portion of nose assembly 802 includes an end portion of positioning rod 110, hollow cutting sheath 116, an end portion of actuation member 118, a visual indicator 812 and ventilation tube 114. Like visual indicator 112, visual indicator 812 includes a pair of base members 824a and 824b spaced apart from each other and that extend from first ends 821a and 821b, bend at second ends 825a and 825b and couple together at 836. The pair of members 824a and 824b form a protruding tab 826 between bends at second ends 825a and 825b and 836. First ends 821a and 821b are configured to mate with and be coupled to positioning rod 110. Visual indicator 812 may be rigid, semi-rigid or flexible and made of, for example, metal, plastic, a thermoplastic polymer, such as PTFE, silicone, rubber, etc. and may be soldered, welded, adhered, or molded to positioning rod 110.

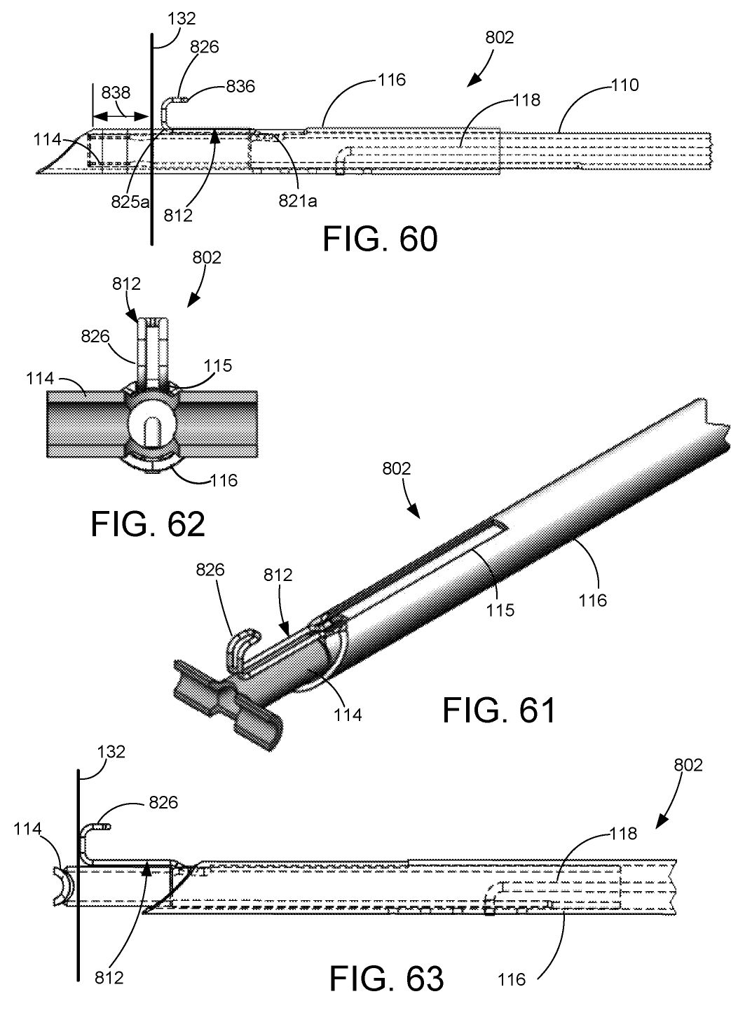

FIG. 60 illustrates a side view of FIG. 57 including further illustrating a position of nose assembly 802 after incising tympanic membrane 132 but before tube deployment and as previously illustrated in FIG. 6 but with a different visual indicator 812. As discussed above, protruding tab 826 of visual indicator 812 protrudes through cutting sheath slot 115 and outwardly from an outer surface of cutting sheath 116. Therefore, at least the width of a portion of visual indicator 812 is small enough to fit through a width of cutting sheath slot 115.

Pair of members 824a and 824b of visual indicator 812 extends distally from first ends 821a and 821b along the cutting sheath slot 115, bends at second ends 825a and 825b and join at 836. As illustrated in FIGS. 57-60, visual indicator 812 includes the same width 827 throughout its entirety. The embodiment of visual indicator 812 illustrated in FIGS. 57-60 is less obstructive of the view of the target membrane than visual indicators 126, 526 and 626 due to the thin shape of tab 826. In addition, visual indicator 826 is less likely to be over inserted compared to visual indicator 626 because the shape of tab 826 provided by bends at second ends 825a and 825b and coupling at 836 provides a guard against over insertion. Second ends 825a and 825b are positioned at a distance 838 from distal cutting edge 113 that is equal to a depth of the incision to be made in target membrane 132 of a patient. Therefore, when in a loaded configuration as illustrated in FIGS. 57-60 and a clinician is piercing and making an incision in the target membrane 132 of a patient using distal cutting edge 113, the clinician manually advances or inserts cutting sheath 116 only up to second ends 825a and 825b as illustrated in FIG. 60 so that tab 826 of visual indicator 812 remains visible to the clinician and proper incision depth is maintained.

FIG. 61 illustrates a perspective view of the end portion of nose assembly 802 of FIG. 57 in a tube deployment configuration according to an embodiment. FIG. 62 illustrates a medial end view of FIG. 61, and FIG. 63 illustrates a side view of FIG. 61 including further illustrating a position of nose assembly 802 after ventilation tube 114 is deployed and anchored in tympanic membrane 132. To deploy and anchor ventilation tube 114 in tympanic membrane 132, pull mechanism 106 (FIG. 2) actuates actuation member 118 to withdraw or retract hollow cutting sheath 116 from membrane 132 and from around ventilation tube 114 The retraction of hollow cutting sheath 116 from around ventilation tube 114 causes ventilation tube 114 to re-form, unfold and anchor in membrane 132 while positioning rod 110 and visual indicator 812 remain in place. After ventilation tube 114 is deployed and anchored, nose assembly 802 including visual indicator 812 and positioning rod 110 is completely withdrawn from the patient’s ear.

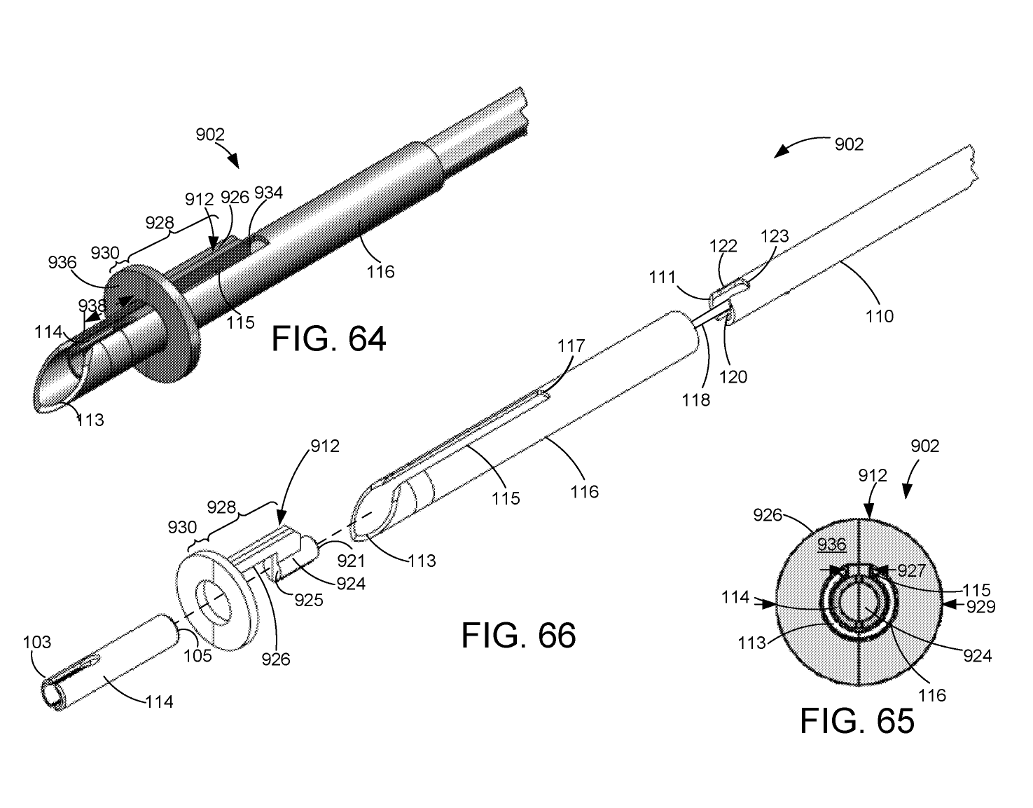

FIG. 64 illustrates a perspective view of an end portion of nose assembly 902 of a tube deploying system according to an embodiment. FIG. 65 illustrates a medial end view of FIG. 64 and FIG. 66 illustrates an exploded perspective view of the end portion of nose assembly 902 of FIG. 64 The illustrated end portion of nose assembly 902 includes an end portion of positioning rod 110, hollow cutting sheath 116, an end portion of actuation member 118, a visual indicator 912 and ventilation tube 114. Like visual indicator 112, visual indicator 912 mates or engages with distal end 111 of positioning rod 110 and includes a base 924 and a tab 926 that protrudes from the base 924. Tab 926 has an elongated portion 929 and a fin portion 930. Elongated portion 928 has width 927 that is less than width 929 of fin portion 930. Fin portion 930 includes a height 933 that is greater than a height 931 of elongated portion 928.

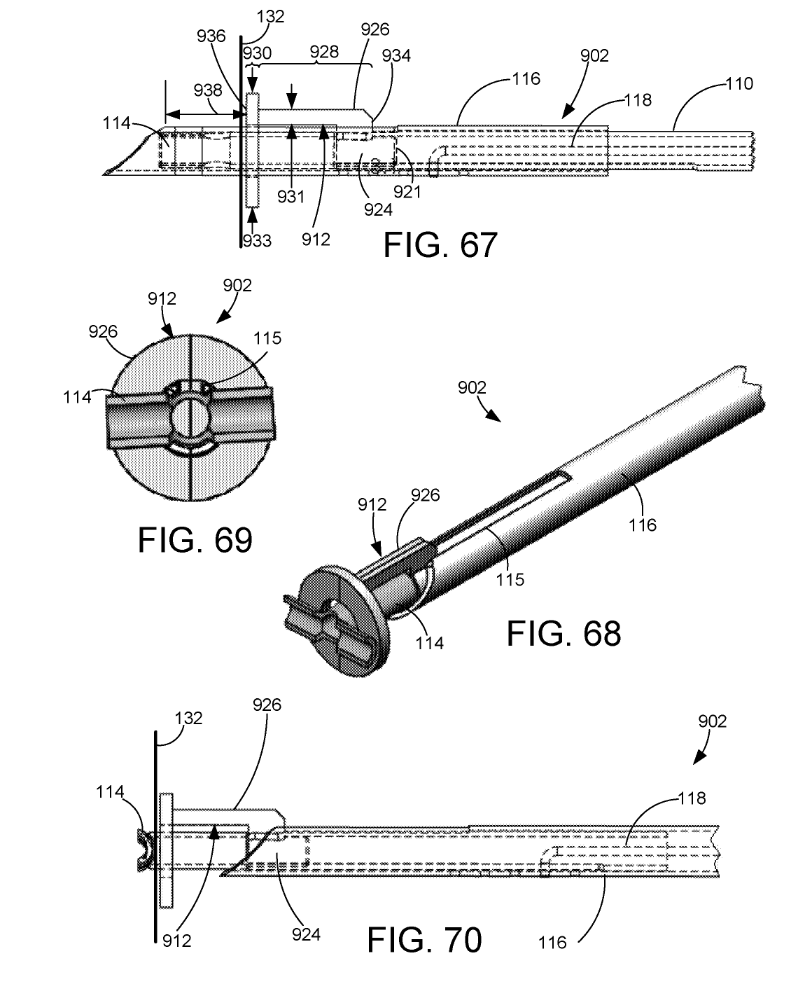

FIG. 67 illustrates a side view of FIG. 64 including further illustrating a position of nose assembly 902 after incising tympanic membrane 132 but before tube deployment as previously illustrated in FIG. 6 but with a different visual indicator. Base 924 of visual indicator 912 includes a first end 921 mated to distal end 111 of positioning rod 110 and a second end 925 positioned inside hollow cutting sheath 116 adjacent to lateral end 105 of ventilation tube 114. Tab 926 of visual indicator 912 protrudes from base 924 through cutting sheath slot 115 and outwardly from an entire circumference of outer surface of cutting sheath 116. Therefore at least a width of elongate portion is small enough to fit through a width of cutting sheath slot 115.

Tab 926 includes a first end 934 and a second end 936. Elongated portion 928 of tab 926 extends distally from first end 934 along the cutting sheath slot 115 and terminates at fin portion 930. Fin portion 930 extends from elongated portion 928 and terminates at second end 936. As illustrated in FIGS. 64-67 and as previously discussed, elongated portion 928 includes a width 927, while fin portion 930 includes a different width 929. Width 929 is greater than width 927. In addition, height 933 of fin portion 930 is greater than height 931 of elongated portion 928. While a full circumference fin portion 930 obstructs the view of cutting edge 113 and therefore target membrane 132, over insertion is difficult or impossible and provides a haptic indication of depth. In addition, a full circumference fin portion 930 keeps cutting edge 113 away from an orifice wall, such as an ear canal, during nose assembly 102 advancement. Second end 936 is positioned at a spaced distance 938 from distal cutting edge 113 of cutting sheath 116 that is equal to a depth of the incision to be made in membrane 132 of a patient. Therefore, when in a loaded configuration as illustrated in FIGS. 64-67 and a clinician is piercing and making an incision in target membrane 132 of a patient using distal cutting edge 113, the clinician manually advances or inserts cutting sheath 116 only up to second end 936 of tab 926 as illustrated in FIG. 67.

FIG. 68 illustrates a perspective view of the end portion of nose assembly 902 of FIG. 64 in a tube deployment configuration according to an embodiment. FIG. 69 illustrates a medial end view of FIG. 68, and FIG. 70 illustrates a side view of FIG. 68 including further illustrating a position of nose assembly 902 after ventilation tube 114 is deployed and anchored in tympanic membrane 132. To deploy and anchor ventilation tube 114 in tympanic membrane 132, pull mechanism 106 (FIG. 2) actuates actuation member 118 to withdraw or retract hollow cutting sheath 116 from tympanic membrane 132 and from around ventilation tube 114. The retraction of hollow cutting sheath 116 from around ventilation tube 114 causes ventilation tube 114 to re-form, unfold and anchor in membrane 132 while visual indicator 912 and positioning rod 110 hold ventilation tube 114 in place. After ventilation tube 114 is deployed and anchored, nose assembly 102 including visual indicator 912 and positioning rod 110 is completely withdrawn from the patient’s ear.

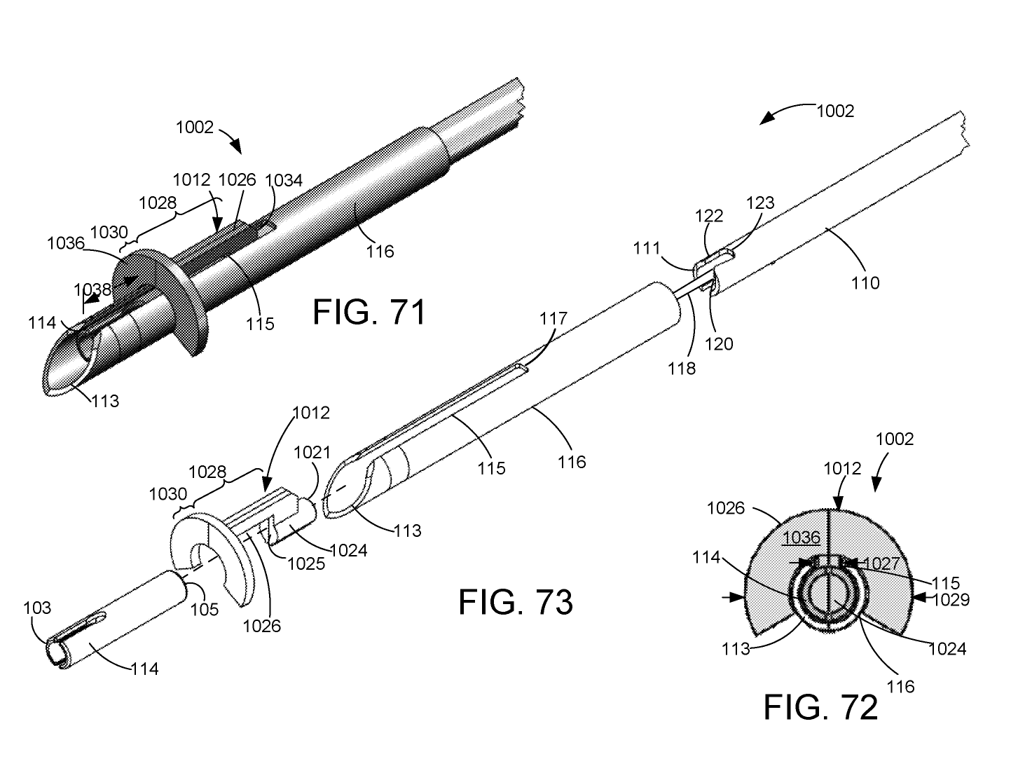

FIG. 71 illustrates a perspective view of the end portion of nose assembly 102 in a loaded configuration according to an embodiment. FIG. 72 illustrates a medial end view of FIG. 71 and FIG. 73 illustrates an exploded perspective view of FIG. 71. The end portion of nose assembly 1002 includes an end portion of positioning rod 110, hollow cutting sheath 116, an end portion of actuation member 118, a visual indicator 1012 and ventilation tube 114. Visual indicator 1012 mates with and is coupled to distal end 111 of positioning rod 110. Like visual indicator 112, visual indicator 1012 mates or engages with distal end 111 of positioning rod 110 and includes a base 1024 and a tab 1026 that protrudes from the base 1024. Tab 1026 has an elongated portion 1028 and a fin portion 1030.

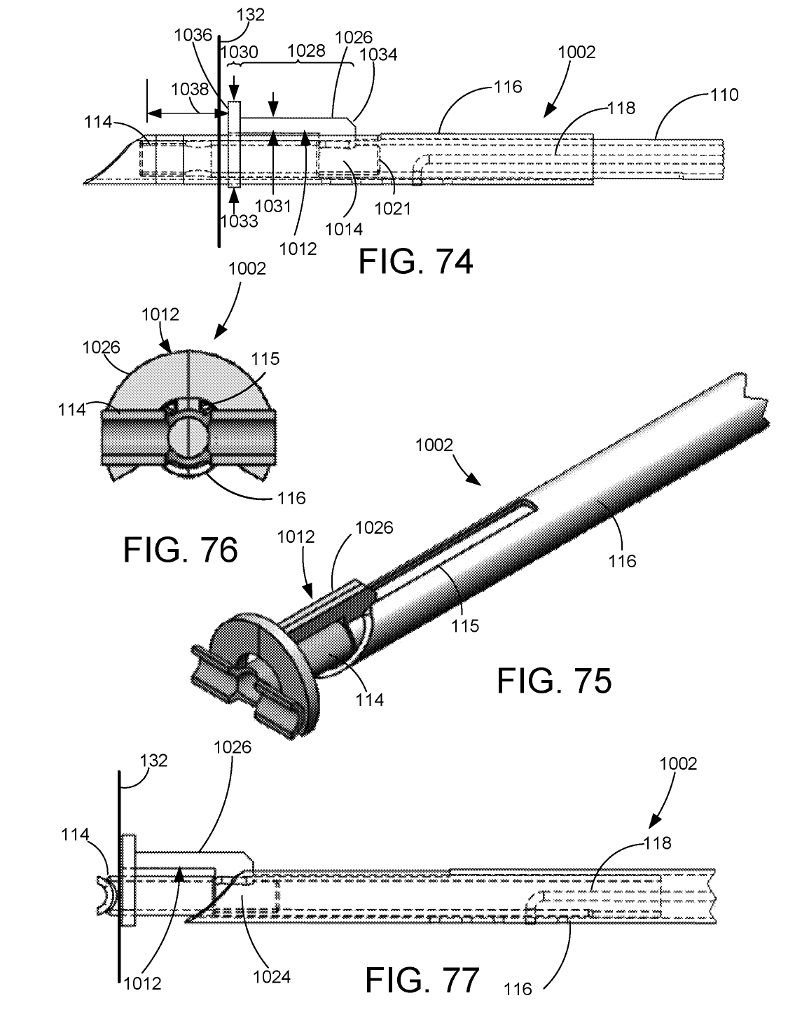

FIG. 74 illustrates a side view of FIG. 71 including further illustrating a position of nose assembly 1002 after incising tympanic membrane 132 but before tube deployment according to an embodiment as previously illustrated in FIG. 6 but with a different visual indicator 1012. Base 1024 of visual indicator 1012 includes a first end 1021 mated to distal end 111 of positioning rod 110 and a second end 1025 positioned inside hollow cutting sheath 116 adjacent to lateral end 105 of ventilation tube 114. Tab 1026 of visual indicator 1012 protrudes from base 1024 through cutting sheath slot 115 and outwardly from a portion of a circumference of an outer surface of cutting sheath. Therefore, at least a width of elongated portion 1028 is small enough to fit through a width of cutting sheath slot 115.

Tab 1026 includes a first end 1034 and a second end 1036. Elongated portion 1028 of tab 1026 extends distally from first end 1034 along the cutting sheath slot 115 and terminates at fin portion 1030. Fin portion 1030 extends from elongated portion 1028 and terminates at second end 1036. As illustrated in FIGS. 71-74 and as previously discussed, elongated portion 1028 includes a width 1027, while fin portion 1030 includes a different width 1029. Width 1029 is greater than width 1027. In addition, height 1033 of fin portion 1030 is greater than height 1031 of elongated portion 1028. While a partial circumference fin portion 1030 partially obstructs the view of cutting edge 113 and therefore target membrane 132, over insertion is difficult to impossible and provides a haptic indication of depth. In addition, a partial circumference fin portion 1030 keeps cutting edge 113 away from an orifice wall, such as an ear canal, during nose assembly 102 advancement. Second end 1036 is positioned at a spaced distance 1038 from distal cutting edge 113 of cutting sheath 116 that is equal to a depth of the incision to be made in membrane 132 of a patient. Therefore, when in a loaded configuration as illustrated in FIGS. 71-74 and when a clinician is piercing and making an incision in membrane 132 of a patient using distal cutting edge 113, the clinician manually advances or inserts cutting sheath 116 only up to second end 1036 of tab 1026 as illustrated in FIG. 74.

FIG. 75 illustrates a perspective view of the end portion of nose assembly 1002 of FIG. 71 in a tube deployment configuration according to an embodiment. FIG. 76 illustrates a medial end view of FIG. 75, and FIG. 77 illustrates a side view of FIG. 75 including further illustrating a position of nose assembly 1002 after ventilation tube 114 is deployed and anchored in tympanic membrane 132. To deploy and anchor ventilation tube 114 in tympanic membrane 132, pull mechanism 106 (FIG. 2) actuates actuation member 118 to withdraw or retract hollow cutting sheath 116 from tympanic membrane 132 and from around ventilation tube 114. The retraction of hollow cutting sheath 116 from around ventilation tube 114 causes ventilation tube 114 to re-form, unfold and anchor in membrane 132 while visual indicator 1012 and positioning rod 110 hold ventilation tube 114 in place. After ventilation tube 114 is deployed and anchored, nose assembly 1002 including visual indicator 1012 and positioning rod 110 is completely withdrawn from the patient’s ear.

Although elements have been shown or described as separate embodiments above, portions of each embodiment may be combined with all or part of other embodiments described above. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A tube deploying system comprising:
a handle having a pull mechanism;
a nose assembly comprising:
a hollow cutting sheath having a distal cutting edge and a cutting sheath slot that extends from the distal cutting edge to a terminating end, wherein in an undeployed configuration the hollow cutting sheath is configured to house a ventilation tube that has a medial end and a lateral end in an undeployed state;
a positioning rod having a distal end located within the hollow cutting sheath;
an actuation member coupling the pull mechanism to the hollow cutting sheath;
a visual indicator coupled to the positioning rod and includes a base and a tab, wherein the tab protrudes through the cutting sheath slot and outwardly from an outer surface of the hollow cutting sheath and distally to an end that is a spaced distance from the distal cutting edge of the hollow cutting sheath in the undeployed configuration; and
wherein in the undeployed configuration the hollow cutting sheath is configured to pierce a membrane of a patient with the distal cutting edge and be manually advanced through the membrane until the end of the visual indicator is adjacent to the membrane; and
wherein in a deployed configuration the actuation member is configured to retract the hollow cutting sheath from around the ventilation tube and along the visual indicator and the positioning rod to leave the ventilation tube in a deployed state in the membrane of the patient.

2. The tube deploying system of claim 1, wherein the positioning rod comprises a first slot that extends from the distal end of the positioning rod to a terminating point and a second slot that is located opposite the first slot, wherein the first slot provides access for the actuation member located internal to the positioning rod to couple to the cutting sheath.

3. The tube deploying system of claim 2, wherein the base of the visual indicator is at least partially surrounded by the positioning rod and wherein the tab protrudes from the base of the visual indicator through the second slot in the positioning rod and the cutting sheath slot and includes a first end and a second end.

4. The tube deploying system of claim 3, wherein a portion of the base of the visual indicator is configured to engage with the first slot in the positioning rod.

5. The tube deploying system of claim 3, wherein the second slot in the positioning rod is defined between a first end located a spaced distance from distal end of positioning rod and an opposing second end and between a first lateral side and an opposing second lateral side, wherein a portion of the base is configured to engage with the second slot in the positioning rod.

6. The tube deploying system of claim 3, wherein the tab further comprises an elongated portion and a fin portion coupled to the elongated portion, wherein the fin portion comprises a height that is greater than a height of the elongated portion.

7. The tube deploying system of claim 6, wherein the fin portion of the tab comprises a plurality of spaced apart grooves that extend along the height of the fin portion, the plurality of grooves are indicative of trimmable portions of the fin portion to adjust a length from the first end to the second end of the tab.

8. A tube deploying system comprising:
a ventilation tube having a medial end and a lateral end;
a hollow cutting sheath having a distal cutting edge and a cutting sheath slot that extends from the distal cutting edge to a terminating end, the hollow sheath is configured to house the ventilation tube;
a positioning rod having a distal end located within the hollow cutting sheath and being adjacent the ventilation tube;
an actuation member coupled to the hollow cutting sheath; and
a visual indicator including a base and a tab that protrudes from the base, wherein the base is located and fixed internal to the positioning rod and wherein the tab protrudes through the cutting sheath slot and outwardly from an outer surface of the hollow cutting sheath, the tab including a first end and a second end that is located distally from the base; and
wherein the hollow cutting sheath is configured to pierce a membrane of a patient with the distal cutting edge and be manually advanced into the membrane up to the second end of the visual indicator; and
wherein the actuation member is configured to retract the hollow cutting sheath from around the ventilation tube and along the visual indicator and the positioning rod to deploy and leave the ventilation tube in the membrane.

9. The tube deploying system of claim 8, wherein the tab of the visual indicator comprises an elongated portion and a fin portion.

10. The tube deploying system of claim 9, wherein a width of the elongated portion and a width of the fin portion are uniform.

11. The tube deploying system of claim 9, wherein the fin portion comprises a height that is greater than a height of the elongated portion.

12. The tube deploying system of claim 8, wherein the tab comprises a varying height.

13. The tube deploying system of claim 8, wherein the positioning rod comprises a first slot that extends from the distal end of the positioning rod to a terminating point and comprises a second slot located opposite the first slot.

14. The tube deploying system of claim 13, wherein the actuation member is located internal to the positioning rod and extends through the first slot of the positioning rod to couple to the hollow cutting sheath.

15. The tube deploying system of claim 13, wherein a portion of the base of the visual indicator engages with the first slot of the positioning rod to couple to the visual indicator to the positioning rod.

16. The tube deploying system of claim 13, wherein the second slot is defined between a first end located a spaced distance from distal end of positioning rod and an opposing second end and between a first lateral side and an opposing second lateral side.

17. The tube deploying system of claim 16, wherein a portion of the base is configured to engage with the second slot in the positioning rod.

18. The tube deploying system of claim 8, wherein the ventilation tube includes a raised alignment feature on an outer wall of the ventilation tube, the raised alignment feature aligns with the cutting sheath slot and a bottom of the tab, the tab being configured to rest on the raised alignment feature.

19. A method of deploying a ventilation tube comprising:
providing a handle having a pull mechanism and a nose assembly coupled to the handle, the nose assembly including a hollow cutting sheath having a distal cutting edge and a cutting sheath slot that extends from the distal cutting edge to a terminating end, a positioning rod having a distal end located within the hollow cutting sheath, an actuation member coupling a pull mechanism to the hollow cutting sheath, a ventilation tube having a medial end and a lateral end and being housed in the hollow cutting sheath and a visual indicator that is coupled to the positioning rod and protrudes through the cutting sheath slot, outwardly from an outer surface of the hollow cutting sheath and distally to an end of the visual indicator that is a spaced distance from the distal cutting edge of the hollow cutting sheath;
manually advancing the nose assembly in an orifice of a patient;
piercing a membrane of the patient with the distal cutting edge of the hollow cutting sheath and inserting the hollow cutting sheath through the membrane for the spaced distance until the end of the visual indicator is adjacent the membrane; and
manually moving the pull mechanism to retract the hollow cutting sheath from around the ventilation tube to deploy the ventilation tube and thereby leave the deployed ventilation tube in the membrane.

20. The method of claim 17, wherein manually moving the pull mechanism to retract the hollow cutting sheath from around the ventilation tube further comprises manually moving the pull mechanism to retract the hollow cutting sheath from around the ventilation tube and along the visual indicator and the positioning rod as the visual indicator and the positioning rod remain in place.
